SEQUENCE LISTING (16 sequences)

SEQ ID NO: 1 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLNDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 2 - protein, 1999 aa, Homo sapiens
MQPPSLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPGCEVNPDNCVSHQCQNGGTCQDGLDTYTCLCPETWTGWDCSEDVDECEAQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 3 - protein, 268 aa, Homo sapiens [sequence omitted]

SEQ ID NO: 4 - protein, 304 aa, Homo sapiens
MKRAHPEYSSSDSELDETIEVEKESADENGNLSSALGSMSPTTSSQILARKRRRGIIEKRRRDRINNSLSELRRLVPSAFEKQGSAKLEKAEILQMTVDHLKMLHTAGGKGYFDAHALAMDYRSLGFRECLAEVARYLSIIEGLDASDPLRVRLVSHLNNYASQREAASGAHAGLGHIPWGTVFGHHPHIAHPLLLPQNGHGNAGTTASPTEPHHQGRLGSAHPEAPALRAPPSGSLGPVLPVVTSASKLSPPLLSSVASLSAFPFSFGSFHLLSPNALSPSAPTQAANLGKPYRPWGTEIGAF

SEQ ID NO: 5 - protein, 337 aa, Homo sapiens
MKRPCEETTSESDMDETIDVGSENNYSGQSTSSVIRLNSPTTTSQIMARKKRRGIIEKRRRDRINNSLSELRRLVPTAFEKQGSAKLEKAEILQMTVDHLKMLQATGGKGYFDAHALAMDFMSIGFRECLTEVARYLSSVEGLDSSDPLRVRLVSHLSTCATQREAAAMTSSMAHHHHPLHPHHWAAAFHHLPAALLQPNGLHASESTPCRLSTTSEVPPAHGSALLTATFAHADSALRMPSTGSVAPCVPPLSTSLLSLSATVHAAAAAATAAAHSFPLSFAGAFPMLPPNAAAAVAAATAISPPLSVSATSSPQQTSSGTNNKPYRPWGTEVGAF

SEQ ID NO: 6 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 7 - protein, 1238 aa, Homo sapiens [sequence omitted]

SEQ ID NO: 8 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 9 - DNA, 18 bp, Artificial sequence
  Synthetic sequence Forward primer
cgagctggacgagaccat

SEQ ID NO: 10 - DNA, 20 bp, Artificial Sequence
  Synthetic sequence Reverse primer
gagccgaactcaagtttcca

SEQ ID NO: 11 - DNA, 18 bp, Artificial Sequence
  Synthetic sequence Forward primer
gaagcacctccggaacct

SEQ ID NO: 12 - DNA, 20 bp, Artificial Sequence
  Synthetic sequence Reverse primer
gtcacctcgttcatgcactc

SEQ ID NO: 13 - DNA, 23 bp, Artificial Sequence
  Synthetic sequence Forward primer
tctacaaccctgaagtgcttgat

SEQ ID NO: 14 - DNA, 21 bp, Artificial Sequence
  Synthetic sequence Reverse primer
caatctgcagacagacactgg

SEQ ID NO: 15 - DNA, 19 bp, Artificial Sequence
  Synthetic sequence Forward primer
agccacatcgctcagacac

SEQ ID NO: 16 - DNA, 19 bp, Artificial Sequence
  Synthetic sequence Reverse primer
gcccaatacgaccaaatcc

(19) United States
(12) Patent Application Publication
(10) Pub. No.: US 2016/0376659 A1
(43) Pub. Date: Dec. 29, 2016

(54) Breast Cancer Biomarker

(71) Applicant: The University of Manchester, Manchester (GB)

(72) Inventors: Robert Clarke, Manchester (GB); Bruno Simões, Manchester (GB); Sacha Howell, Manchester (GB); Rachel Eyre, Sale (GB); Goran Landberg, Manchester (GB)

(21) Appl. No.: 14/748,584

(22) Filed: Jun. 24, 2015

(51) Int. Cl. C12Q 1/68; C12Q 1/32; A61K 31/565; G01N 33/574; A61K 31/55; A61K 31/138

(52) U.S. Cl. CPC C12Q 1/6886; A61K 31/55; A61K 31/138; A61K 31/565; G01N 33/57415; C12Q 1/32; C12Q 2600/158; C12Q 2600/118; C12Q 2600/106; G01N 2333/90203

(57) ABSTRACT

The invention relates to cancer, and more specifically to breast cancer, and particularly to estrogen receptor positive breast cancer that is resistant to endocrine therapy. The invention provides methods for selecting subgroups of subjects for treatment, and methods of treatment of a subgroup of subjects.

### FIELD OF THE INVENTION

The invention relates to cancer, and more specifically to breast cancer, and particularly to estrogen receptor positive breast cancer. The invention provides methods for selecting subgroups of subjects for treatment, and methods of treatment of a subgroup of subjects.

Incorporated by reference herein in its entirety is the Sequence Listing entitled “sequence_listing_ST25,” created Jul. 7, 2015, size of 58 kilobytes.

### BACKGROUND TO THE INVENTION

Around 75% of cases of breast cancer express the estrogen receptor (ER), and respond to the anti-proliferative effects of endocrine therapy. Anti-estrogen drugs include selective ER modulators (SERMs) such as tamoxifen and pure anti-estrogens such as fulvestrant. Other drugs include aromatase inhibitors such as letrozole and anastrozole.

However, hormone-responsive breast cancers frequently develop resistance to hormonal therapies. The potential involvement of breast cancer stem cells (CSCs) in endocrine resistance makes it imperative to understand the cellular signaling pathways that could be targeted to eradicate breast CSCs and, therefore, provide long term disease-free survival. One strong candidate for is the Notch pathway, which is known to be activated in breast CSCs.

Approaches to target estrogen receptor (ER) signaling in breast cancer (BC) have had a profound effect on survival in women with early and advanced disease (Ali and Coombes, 2002). However, resistance to established therapies such as selective ER modulators (SERMs e.g. tamoxifen), selective ER downregulators (SERDs e.g. fulvestrant) and the aromatase inhibitors (AIs e.g. letrozole, anastrozole, exemestane) is seen in 50-60% of early BC cases and develops in almost all patients with advanced disease (EBCTCG 2011; Palmieri et al., 2014). Approaches to reverse endocrine resistance are a topic of intense research, with large potential clinical gains, given the increasing incidence of ER positive BC (Palmieri et al., 2014).

Al-Hajj and colleagues (2003) were the first to show that CD44⁺/CD24^{-/low}/ESA⁺/lineage⁻ BC cells were enriched for tumor-initiating cells and were capable of recapitulating the original tumor phenotype when transplanted into non-obese diabetic/severe combined immunodeficient (NOD/SCID) mice. In vitro functional assays for BC stem cell (BCSC) activity include aldehyde dehydrogenase 1 (ALDH1) enzyme activity and the capacity to form clonogenic spheres in suspension culture (Ginestier et al., 2007, Pece et al., 2010). Evidence suggests that tumor-initiating or cancer stem-like cells (CSCs) are responsible for tumor recurrence after chemo- and endocrine therapy (Li et al., 2008; Creighton et al., 2009). Several groups, including our own, have demonstrated that this enriched BCSC population is ER negative/low and resistant to the direct effects of endocrine therapy (Simões et al., 2011; Haughian et al., 2012; Piva et al., 2014, Harrison et al., 2013).

We have previously shown that aberrant Notch activation transforms normal breast cells, is found in pre-invasive and invasive human BC, and in DCIS correlates with early recurrence (Stylianou et al., 2006; Farnie et al., 2007). Moreover, we reported that inhibition of Notch signaling, particularly NOTCH4 receptor, reduced BCSC activity (Harrison et al., 2010).

### SUMMARY OF THE INVENTION

Here, using primary patient-derived ER+ BC samples and xenografts (PDX), we report that short-term treatment with endocrine therapies enriches for NOTCH4-regulated BCSCs, suggesting these effects are not through genetic selection or evolution. However, combining endocrine therapies with Notch pathway inhibition reverses the increase in BSCSs. Furthermore, we show that ALDH1 expression and NOTCH4 activation in human primary tumors are predictive of resistance to endocrine treatments. Finally, combination therapy reduces BCSC activity in long-term acquired resistant PDX tumors. Thus, we propose that inhibiting Notch signaling will help overcome endocrine therapy resistance and recurrence in ER+ BC. As described herein, Notch, and particularly Notch4, may be targeted to diagnose, predict, or reverse, resistance to endocrine therapy, and particularly endocrine resistant ER+ breast cancer.

The present invention provides a method comprising determining the level of ALDH1 in a subject with estrogen receptor positive (ER+) breast cancer and selecting the subject of treatment with a INS if the level of ALDH1 is elevated. The method may further comprise administering endocrine therapy to the subject, such that endocrine therapy is administered in combination with an Inhibitor of NOTCH Signaling (INS).

The subject may already have exhibited resistance to endocrine therapy, or may be suspected of having developed resistance to endocrine therapy. The subject may already be undergoing endocrine therapy, or may previously have been treated with endocrine therapy. The endocrine therapy may be therapy with tamoxifen or fulvestrant or tamoxifen and fulvestrant.

The subject may have late stage breast cancer, or metastatic breast cancer. Alternatively, the subject may have early stage breast cancer. In certain aspects, the INS administered to the subject is a gamma secretase inhibitor (GSI), such as RO4929097 (Roche).

In certain methods disclosed herein, the subject is selected for treatment of ALDH1 level is elevated relative to a subject known to be responsive to endocrine therapy. Alternatively, the subject may be selected for treatment if ALDH1 level is elevated relative to the level of ALDH1 in the same subject at an earlier date, such as prior to undergoing endocrine drug therapy, or shortly after commencing endocrine drug therapy.

Certain methods disclosed herein involve selecting a subject undergoing endocrine therapy to treat ER+ breast cancer; determining the level of ALDH1 in the subject; and selecting the subject for treatment with INS if the level of ALDH1 is determined to be elevated. The treatment may be supplementary to the endocrine therapy. The method may further comprise a step of administering the INS to the subject.

Other methods disclosed herein include methods for predicting the outcome of hormone breast cancer therapy in a subject. Such methods may comprise determining the level of ALDH1 in the subject, wherein a high level of ALDH1 indicates a poor response to treatments with an endocrine drug, such as a SERM or SERD drug, and selecting the subject for treatment with breast cancer therapy which is not an endocrine therapy, or selecting the subject for treatment with a INS in addition to endocrine therapy. Where an INS is administered in addition to endocrine therapy, the endocrine therapy may be the same endocrine therapy that the subject was on prior to determining the ALDH1 level, or a different therapy.

Other methods disclosed herein include methods of treatment comprising administering to a subject determined to have ER+ breast cancer and elevated ALDH1, an effective amount of an endocrine therapy and an INS.

Also disclosed herein are pharmaceutical compositions comprising an endocrine therapy such as a SERD or SERM drug, and an INS. Thus, the pharmaceutical composition is a combination of at least two agents. In some cases, the drug is tamoxifen or fluvestrant, and the INS is RO49290967 or a derivative thereof.

Other methods disclosed herein include methods for diagnosing resistance to endocrine therapy in a subject with ER+ breast cancer, the method comprising determining the level of ALDH1 in the subject, wherein an elevated level of ALDH1 is indicative that the subject is resistant to endocrine therapy.

Also described is the use of a INS to switch ER+ breast cancer cells that are resistant to endocrine therapy susceptible to endocrine therapy. Cells may become resistant to endocrine therapy after prolonged exposure to such therapy, and the INS may reverse such resistance to make the cells susceptible again.

Also disclosed is a method for treating cancer that has acquired resistance to hormone therapy involving revising the acquired resistance by administering an inhibitor of Notch4.

Also disclosed is a method comprising determining the level of ALDH1 in a subject determined to have ER+ breast cancer, administering endocrine therapy to the subject; further determining the level of ALDH1 in the subject after administration of the endocrine therapy; and if the level of ALDH1 in the subject is determined to have increased, selecting the subject for treatment with INS. In some cases, the method further involves the step of administering INS to the subject.

The invention also provides use of a Notch4 inhibitor to increase the susceptibility of ER+ breast cancer cells to endocrine therapy.

Another aspect of the present disclosure is a method comprising determining the level of expression of one or more of genes selected from NOTCH4, HES1, HEY1 and HEY2 in a subject with ER+ breast cancer and, if said one or more genes is elevated, selecting the subject for treatment for combination therapy with an INS and an endocrine therapy.

### BRIEF DESCRIPTION OF THE DRAWINGS

FIG. 1A, 1B, 1C, 1D, 1E, 1F, 1G, 1H: Tamoxifen or fulvestrant treatments of ER+ patient-derived samples and patient derived xenografts (PDXs) selectively enrich for cells with cancer stem cell properties. High BCSC frequency is associated with worse outcomes for tamoxifen treated BC patients. FIG. 1A) Mammosphere self-renewal of freshly isolated ER+ early and metastatic patient-derived samples. Primary mammospheres cultured in the presence of ethanol (Control) or 10⁻⁶ M 4-hydroxy-tamoxifen were dissociated and replated in secondary mammosphere suspension culture for a further 7-9 days to measure self-renewal of mammosphere-initiating cells treated in the first generation. P-value calculated with Wilcoxon signed-rank test. FIG. 1B) Representative micrographs of metastatic BC cells before FACS analysis of ALDH1 enzymatic activity (ALDEFLUOR assay). ALDH-positive cells were discriminated from ALDH-negative cells using the ALDH inhibitor, DEAB. FIG. 1C) Percentage of ALDH-positive cells in 9 ER+ metastatic BC patient-derived samples. Cells were grown in adherence with ethanol (Control), Tamoxifen (10⁻⁶M) or Fulvestrant (10⁻⁷ M) for 7-9 days. Arrows indicate fold-change greater than 20% compared to control. FIGS. 1D, 1E, 1F, and 1G) Early (HBC×34) and metastatic (BB3RC31) BC estrogen-dependent patient-derived xenograft tumors treated in vivo for 14 days with tamoxifen (10 mg/kg/day, oral gavage) or fulvestrant (200 mg/kg/week, subcutaneous injection). FIG. 1E) Representative micrographs and quantification of Ki67 expression determined by immunohistochemistry. FIG. 1F) Percentage of mammosphere formation efficiency (MFE). FIG. 1G) ALDH-positive cells (%) determined using the ALDEFLUOR assay. FIG. 1H) Aldehyde dehydrogenase 1 (ALDH1) expression was assessed by immunohistochemistry in breast tumor epithelial cells and the percentage of positive cells was scored. Representative micrographs of ALDH-high and -low epithelial expression are shown. Kaplan-Meier curves represent cumulative survival for ALDH^{lo} population and ALDH^{hi} population of a cohort of 322 pre-menopausal ER+ BC patients who participated in a randomised trial of 2 years of adjuvant tamoxifen treatment versus no systemic treatment (control). Vertical bars on survival curves indicate censored cases. P-values are based on a Log Rank (Mantel-Cox) test of equality of survival distributions. Scale bars=100 μm. Data are represented as mean±SEM. * p<0.05, ** p<0.01 FIG. 2A, 2B, 2C, 2D: Tamoxifen or fulvestrant treatments up-regulate Notch target genes in patient-derived samples, PDXs and cell lines. Endocrine-resistant BC cells display increased activation of Notch signaling. FIG. 2A, 2B, 2C) Expression of Notch target genes HEY1 and HES1 was assessed by quantitative real-time PCR analysis and compared to control to determine fold change. FIG. 2A) Metastatic BC patient-derived cells were treated for 7-9 days with ethanol (Control), Tamoxifen (10⁻⁶ M) or Fulvestrant (10⁻⁷ M) and a correlation between fold change of expression of HEY1 and HES1 and fold change of percentage of ALDH+ cells is shown. FIG. 2B) Early (HBC×34) and metastatic (BB3RC31) BC PDXs: the effect of in vivo treatment for 14 days with tamoxifen (10 mg/kg/day, oral gavage) or fulvestrant (200 mg/kg/week, subcutaneous injection) on HEY1 and HES1. FIG. 2C) MCF-7, T47D and ZR-75-1 cells: the effect of treatment with 10⁻⁶ M tamoxifen (red bar) and 10⁻⁷ M fulvestrant (blue bar) for six days on HEY1 and HES1. FIG. 2D) Expression of HEY1 and HES1 in endocrine-resistant cells compared to the parental MCF-7 cells (two left hand panels). Notch transcriptional activity in endocrine-resistant cells compared to control was determined by relative firefly luciferase activity of 10×CBF1 reporter (right hand panel).

Data are represented as mean±SEM. * p<0.05; ** p<0.01

FIG. 3A, 3B, 3C, 3E, 3F, 3G: JAG1-NOTCH4 receptor signaling drives Notch activity in endocrine-resistant BC and predicts for resistance to tamoxifen treatment. FIG. 3A, FIG. 3B, FIG. 3C) NOTCH4, HES1 and JAG1 protein expression levels determined by Western Blot in MCF-7 endocrine-resistant cells; FIG. 3(A), MCF-7 estrogen-sensitive cells FIG. 3(B) and metastatic (BB3RC31) BC PDX FIG. 3(C). β-actin was used as a reference for the loading control. FIG. 3D, FIG. 3E) NOTCH4, HES1, HEY1 and HEY2 genes in ER+ primary tumors from tamoxifen-treated FIG. 3(D) or untreated FIG. 3(E) patients are co-expressed in the heatmap ranked from left to right using the four gene signature. Colours are log 2 mean-centered values, Red=high, Green=low. All significant cut-points (p<0.05) are shown in grey. Kaplan Meier analysis using the optimum cut-point (dashed white line) demonstrates that elevated expression of the Notch genes is significantly associated with an increased rate of distant metastasis FIG. 3(D) and overall survival FIG. 3(E). Vertical bars on survival curves indicate censored cases. P-values are based on a Log Rank (Mantel-Cox) test FIG. 3(F) Tamoxifen and fulvestrant up-regulation of Notch target genes HEY1 and HES1 FIG. 3(G) Gamma secretase inhibitors DAPT and DBZ inhibit NOTCH1 activity but not NOTCH4 activity.

FIG. 4A, 4B, 4C, 4D, 4E: Gamma-secretase inhibitor RO4929097 targets NOTCH4 signaling. NOTCH4 receptor is needed for anti-estrogen CSC activity and confers anti-estrogen insensitivity. FIG. 4A) NOTCH4 intracellular domain expression levels determined by Western Blot in MCF-7 endocrine-resistant cells treated with GSI RO4929097 or control DMSO for 72 h. FIG. 4B, 4C) Expression of HEY1 and HES1 and Notch transcriptional activity (10×CBF1-luciferase reporter) are inhibited by GSI RO4929097 (72 h) in endocrine-resistant cells. FIG. 4D) MCF-7 shNOTCH4 cells pre-treated in adherence with 10⁻⁶ M tamoxifen (red bar) and 10⁻⁷ M fulvestrant (blue bar) in the presence of doxycycline (1 μg/mL; hatched bars) or water (filled bars) for 6 days. Fold change in MFE and ALDH-positive cells after treatment with doxycycline compared to the respective cells treated with control water. FIG. 4E) NICD4 rescues tamoxifen- or fulvestrant-inhibited growth: cell number (using SRB assay) of MCF-7 overexpressing NICD4-GFP or GFP (y axis) incubated with tamoxifen or fulvestrant for 1, 3 and 5 days (x axis) compared to the respective cell line treated with control ethanol. P-values for the 5 days treatment. Data are represented as mean±SEM. * p<0.05; ** p<0.01

FIG. 5A, 5B, 5C, 5D, 5E, 5F, 5G, 5H: NOTCH4 inhibition using RO4929097 abrogates tamoxifen and fulvestrant enrichment of cancer stem cell activities. FIG. 5A, 5B, 5C) Early (HBC×34) and metastatic (BB3RC31) PDX tumors treated in vivo for 14 days with tamoxifen (10 mg/kg/day, oral gavage) or fulvestrant (200 mg/kg/week, subcutaneous injection) in the presence or absence of the NOTCH4 inhibitor RO4929097 (3 mg/kg/day, oral gavage). FIG. 5A) MFE (%). FIG. 5B) Percentage of ALDH-positive cells. FIG. 5C) Secondary tumor formation. 100,000 cells of metastatic (BB3RC31) PDX were re-implanted subcutaneously in NSG mice with 90-day slow release estrogen pellets. Tumor growth (>100 mm3) was determined at day 90 after cell injection. FIG. 5D, 5E) MCF-7, T47D and ZR-75-1 cells were pre-treated in adherence with 10⁻⁶ M tamoxifen (red bar) and 10⁻⁷ M fulvestrant (blue bar) with RO4929097 (10 μM; hatched bars) or DMSO (filled bars) for 72 hours. MFE, FIG. 5(D) and percentage of ALDH-positive cells FIG. 5(E) were assessed after pre-treatments. FIG. 5F, FIG. 5G, FIG. 5H) MCF-7 cells were pre-treated in adherence for six days in the presence of RO4929097 (10 μM; hatched bars) or DMSO (filled bars). (FIG. 5F) In vivo experiments were carried out in NSG mice with 90-day slow release estrogen pellets. Tumor growth (>100 mm3) was assessed at day 60 and is represented as mice positive for growth/mice tested for each cell number tested. ELDA analysis of tumor initiating cell frequency is shown. FIG. 5G) Expression of HEY1 and HES1 by real-time PCR was compared to the control. FIG. 5H) Hes1 protein expression levels determined by Western Blot. Data are represented as mean±SEM. p-values refer to hatched bars compared to filled control bars. * p<0.05; ** p<0.01

FIG. 6A, 6B, 6C, 6D, 6E, 6F: HBC×22 and HBC×34 tamoxifen-resistant PDXs express high levels of HES1. NOTCH4 inhibitor RO4929097 targets cancer stem-like cells in tamoxifen-resistant PDXs. FIG. 6A) Representative micrographs and quantification of HES1 expression determined by immunohistochemistry. FIG. 6B, 6C, 6D) HBC×22 and HBC×34 tamoxifen-resistant PDXs treated in vivo for 14 days in the presence or absence of the gamma-secretase inhibitor RO4929097 (10 mg/kg/day, oral gavage). FIG. 6B) MFE (%). FIG. 6C) Percentage of ALDH-positive cells. FIG. 6D) Representative FACS plots of ALDEFLUOR assay. ALDH-positive cells were discriminated from ALDH-negative cells using the ALDH inhibitor, DEAB. FIG. 6E) Model depicting NOTCH4-JAG1 signaling as a driving force of Notch activity in endocrine-resistant breast cancer. FIG. 6F) Diagram suggesting that endocrine therapies do not target BCSCs, and emphasizing the need of targeting residual drug-resistant cells to eliminate all cancer cells and prevent long-term recurrences of ER+ BC.

FIG. 7A, 7B, 7C, 7D, 7E: FIG. 7A) Mammosphere formation efficiency (MFE) of freshly isolated ER+ early and metastatic patient-derived samples cultured in the presence of ethanol (Control) or 10⁻⁶ M 4-hydroxytamoxifen. Mammosphere data for each individual patient sample is represented. FIG. 7B) Primary mammospheres were dissociated and replated in secondary mammosphere suspension culture for a further 7-9 days to measure self-renewal of mammosphere-initiating cells treated in the first generation. FIG. 7C) Early (HBC×34) and metastatic (BB3RC31) xenograft tumor sections stained for ER, PR, HER2 and Ki67 by immunohistochemistry. Scale bars=100 μm. FIG. 7D) Ovariectomized mice and administration of estrogen in the drinking water (8 μg/ml) were used to perform an estrogen dependence test of metastatic BB3RC31 PDX. Graph shows tumor formation and size at 120 days after implantation. Tumor formation was determined by counting tumors greater than 100 mm3 (tumors bigger than 100 mm3 are represented by solid forms and tumors smaller than 100 mm3 are represented by hollow forms). Mean of each group is represented by horizontal bar. OveX—Ovariectomized; E2—Estrogen FIG. 7E) ALDH1A1 expression from ER+ BC after presurgical treatment with fulvestrant (n=22) compared to ALDH1A1 expression before treatment (Patani et al., 2014). Data is represented as log 2 fold change. P-value calculated with Wilcoxon Mann-Whitney test.

FIG. 8A, 8B, 8C, 8D, 8E, 8F, 8G: FIG. 8A, 8B, 8C, 8D) MCF-7, T47D and ZR-75-1 cells were pre-treated in adherence with 10⁻⁶ M tamoxifen and 10⁻⁷ M fulvestrant for six days. FIG. 8B), percentage of ALDH-positive cells FIG. 8C) and in vivo tumor growth by 100,000 MCF-7 cells FIG. 8D) were assessed after pre-treatments. In vivo experiments were carried out in Nude female mice and administered with 8 ug/ml of 17-beta oestradiol in drinking water. Tumor growth (>100 mm³) at day 90 is shown. FIG. 8E) ER positive cells as a percentage of total cell population in parental and aldefluor positive (ALDH+) cells were determined in MCF7 and T47D cell lines by immunocytochemistry. Right hand panel shows representative micrographs of ER staining in MCF-7 cells. FIG. 8F) Fold change of percentage of ALDH+ cells and fold change of expression of Hes1 and Hey1 in BC patient-derived cells treated for 7-9 days with Tamoxifen or Fulvestrant compared to control. FIG. 8G) Expression of HES1 assessed by quantitative real-time PCR analysis from MCF-7 ALDH+ sorted cells compared to expression in ALDH− cells to determine fold change. MCF-7 cells were treated with tamoxifen, fulvestrant or control for six days before ALDH sorting. Scale bars=100 μm. Data are represented as mean±SEM. * p<0.05, ** p<0.01

FIG. 9A, 9B: FIG. 9A) Representative FACS plots of ALDEFLUOR assay showing percentage of ALDH-positive cells in MCF-7 tamoxifen-resistant and fulvestrant-resistant cells. ALDH-positive cells were discriminated from ALDH-negative cells using the ALDH inhibitor, DEAB. Fold change of ALDH-positive cells in MCF-7 endocrine-resistant cells compared to the parental MCF-7 cells is shown. FIG. 9B) Percentage of MFE and ALDH-positive cells in HBC×34 endocrine-sensitive (Parental) and HBC×34 tamoxifen-resistant (TAMR) PDXs.

Data are represented as mean±SEM. * p<0.05, ** p<0.01

FIG. 10A, 10B, 10C: FIG. 10A) Notch receptors and Notch ligands protein expression levels determined by Western Blot in MCF-7 endocrine-resistant cells. β-actin was used as a reference for the loading control. FIG. 10B) Early (HBC×34) and metastatic (BB3RC31) PDXs tumor size variation over 14 days in vivo treatments with tamoxifen (10 mg/kg/day, oral gavage) or fulvestrant (200 mg/kg/week, subcutaneous injection) in the presence or absence of the gamma-secretase inhibitor RO4929097 (3 mg/kg/day, oral gavage). Tumor size was determined every 3-4 days and fold change was calculated by dividing the tumor size by the size of the respective tumor at day 0. FIG. 10C) structure of RO4929097 (Roche).

FIG. 11: Quantification of Ki67, ER and PR expression determined by immunohistochemistry. Early (HBC×34) and metastatic (BB3RC31) PDX tumors treated in vivo for 14 days with tamoxifen (10 mg/kg/day, oral gavage) or fulvestrant (200 mg/kg/week, subcutaneous injection) in the presence or absence of the gamma-secretase inhibitor RO4929097 (3 mg/kg/day, oral gavage). Data are represented as mean±SEM. * p<0.05; ** p<0.01

FIG. 12: Graphs representing tumor size at day 60 after cell injection for each cell number tested. MCF-7 cells were pre-treated in adherence for six days in the presence of RO4929097 (10 μM) or DMSO. Experiments (N=4 per condition) were carried out in NOD SCID IL2gammaR knock out (NSG) mice injected subcutaneously with 10.000, 1.000, 100 and 10 cells. 90-day slow release estrogen pellets were implanted sub-cutaneously into mice two days before cell injection (0.72 mg, Innovative Research of America). Positive tumor growth was assessed by determining the mice bearing a tumor greater than 100 mm3 and is represented as mice positive for growth/mice tested.

FIG. 13: Characteristics of “early” patient-derived tumors used in the study. PBC—primary breast cancer.

FIG. 14: Characteristics of “late” metastatic endocrine therapy-treated patient-derived tumours used in the study. Met BC—metastatic breast cancer.

### DETAILED DESCRIPTION

Breast cancers (BCs) typically express estrogen receptors (ER) but frequently exhibit de novo or acquired resistance to hormonal therapies. Here, we show that short-term treatment with anti-estrogens tamoxifen and fulvestrant decrease cell proliferation but increase BC stem cell (BCSC) activity through NOTCH4 receptor activation both in patient-derived samples and xenograft (PDX) tumors. In support of this novel mechanism of resistance, we demonstrate that high ALDH1 predicts resistance in women treated with tamoxifen. Also, a NOTCH4/HES/HEY gene signature predicts for a poor response/prognosis in ER+ patient cohorts. Targeting of NOTCH4 reverses the increase in Notch and BCSC activity induced by anti-estrogens. Importantly, in PDX tumors with acquired tamoxifen resistance, NOTCH4 inhibition reduced BCSC activity.

We observe that the anti-estrogens tamoxifen and fulvestrant increase the percentage of breast CSCs in vivo in ER-positive patient-derived xenograft (PDX) tumours by determining the mammosphere formation efficiency, ALDH enzymatic activity and tumour initiation capacity after treatment. We found that both tamoxifen and fulvestrant increase Notch transcriptional activity via Notch4 and up-regulate Notch target genes, which correlate with ALDH activity. We find that both ALDH1 protein staining and a Notch4 gene signature predict tamoxifen resistance in patients suggesting this is of clinical importance. Finally, we used a gamma-secretase inhibitor (RO4929097), which inhibits Notch activity, to abrogate the CSC increase induced by anti-estrogens. Our results thus far suggest that combining standard endocrine therapies with drugs targeting Notch signaling will be effective in overcoming endocrine therapy resistance of ER-positive breast cancer.

Thus, we establish that BCSC and NOTCH4 activities predict both de novo and acquired tamoxifen resistance and that combining endocrine therapy with targeting NOTCH4 overcomes resistance in human breast cancers.

### Cancer Stem Cells

Cancer stem cells (CSCs) may also be referred to as cancer stem-like cells or tumor-initiating cells. Such cells may have the ability to give rise to all cell types found in a particular type of cancer. Cancer stem cells may be tumorigenic, and have the ability to form a tumor, such as by cell division and self-renewal. Cancer stem cells have been suggested to persist in tumors, and to be associated with relapse and metastasis of cancers.

Several groups have demonstrated colonies grown in vitro from cancer stem-like cells of human breast cancer, including ESA+/CD44+/CD24 cells (Al-Hajj et al; PNAS 2008; Fillmore & Kuperwasser, BCR, 2008; and Harrison et al; Cancer Res. 2010) and ALDHI+/ALDEFLOUR+ cells (Ginestier et al, Stem Cell, 2008).

Cancer stem-like activity for breast cancer stem cells may be measured using mammosphere colonies in vitro and/or tumors in vivo.

Cancer stem cells may be identified and enriched using strategies for identifying normal stem cells, such as antibody labelling and Fluorescence activated cell sorting (FACS). In some cases, cancer stem cells may be identified using the ALDEFLUOR™ fluorescence reagent system in which stem and progenitor cells are isolated based on their expression of the enzyme aldehyde hydrogenase (ALDH) rather than cell surface phenotype. In some cases, cancer stem cells are identified by Side Population (SP) analysis.

### Biomarkers

The present invention particularly relates to biomarkers. Biomarkers, or biological markers, are measurable indicators of a biological state or condition. Biomarkers herein include ALDH1, NOTCH, particularly NOTCH4, HES1, HEY1 and HEY2. These biomarkers may be analyzed alone or in combination to provide information about whether or not a subject will respond to endocrine therapy.

### ALDH1

Aldehyde dehydrogenase 1 is a detoxifying enzyme responsible for the oxidation of intracellular aldehydes. The protein encoded by this gene belongs to the aldehyde dehydrogenase family. Aldehyde dehydrogenase is the next enzyme after alcohol dehydrogenase in the major pathway of alcohol metabolism. There are two major aldehyde dehydrogenase isozymes in the liver, cytosolic and mitochondrial, which are encoded by distinct genes, and can be distinguished by their electrophoretic mobility, kinetic properties, and subcellular localization. This gene encodes the cytosolic isozyme. Studies in mice show that through its role in retinol metabolism, this gene may also be involved in the regulation of the metabolic responses to high-fat diet (provided by RefSeq, March 2011). It is also known as Retinal dehydrogenase 1 (RALDH1), and ALDH-E1, deposited as UniProt P00352-AL1A1_HUMAN, and GenBank accession: AAR92229.1 (GI:40807656)(SEQ ID NO:1).

ALDH may have a role in early differentiation of stem cells, through its role in oxidizing retinol to retinoic acid. It has been shown that murine and human hematopoietic and neural stem and progenitor cells have a high ALDH activity. Increased ALDH activity has also been found in stem cell populations in multiple myeloma and acute myeloid leukemia (AML). ALDH activity may thus provide a common marker for both normal and malignant stem and progenitor cells (see Ginestier et al., 2007).

As disclosed herein, the level of ALDH1 may correlate with resistance to endocrine therapy in ER+ cancers. That is, high expression of ALDH1 may indicate that an individual with ER+ cancer may be resistant to endocrine therapy for the ER+ cancer. ALDH1 may be used as a biomarker on its own, or in combination with one or more of NOTCH, NOTCH4, HES1, HEY1 or HEY2.

### NOTCH

Notch signaling is an evolutionarily conserved pathway in multicellular organisms that regulates cell fate determination during development and maintains adult tissue homeostasis. Mammals possess four different notch receptors, NOTCH1, NOTCH2, NOTCH3 and NOTCH4. The notch receptor is a single-pass transmembrane receptor protein. It is a hetero-oligomer composed of a large extracellular portion, which associates in a calcium dependent, non-covalent interaction with a smaller piece of the notch protein composed of a short extracellular region, a single transmembrane pass and a small intracellular region.

In mammals, ligands for the NOTCH receptors include Delta-like ligands (DLL1, DLL3, DLL4) and Jagged ligands (JAG1 and JAG2). Upon ligand binding, the extracellular domain is cleaved away from the intracellular domain by TNFa ADAM metalloprotease converting enzyme. The extracellular domain remains bound to the ligand and the complex is recycled by the cell. In the signal receiving cell, y-secretase releases the intracellular domain from the transmembrane domain, allowing it to translocate to the nucleus, associated with the CSL transcription factor complex, resulting in activation of a variety of target genes including Myc, p21 and the HES family. Researchers have linked aberrant Notch signaling to a variety of human diseases, including cancers.

Notch4 (GenBank AAC63097.1; GI1841543)(SEQ ID NO:2), also known as Neurogenic locus notch homolog protein 4, is a divergent member of the Notch family of receptors that is primarily expressed in the vasculature. It has previously been reported to have a role in regulating breast cancer stem cell activity (Harrison et al 2010). As disclosed herein, Notch4 expression may correlate with resistance to endocrine therapy in ER+ cancers. That is, high expression of Notch4 may indicate that an individual with ER+ cancer may be resistant to endocrine therapy for the ER+ cancer. Notch may be used alone, or in combination with one or more of ALDH1, HES1, HEY1 or HEY2.

HES1 (GenBank CAA68857.1; GI: 1655594)(SEQ ID NO:3), also known as hairy and enhancer of split-1 is a nuclear protein that suppresses transcription. It is a bHLH transcription factor known to regulate its own expression. As disclosed herein, HES1 expression may correlate with resistance to endocrine therapy in ER+ cancers. That is, high expression of HES1 may indicate that an individual with ER+ cancer may be resistant to endocrine therapy for the ER+ cancer. HES1 may be used alone, or in combination with one or more of ALDH1, HES1, HEY1 or HEY2.

HEY1 (GenBank CAB75715.1; GI: 7018332)(SEQ ID NO:4) also known as Hairy/enhancer of split related with YRPW motif protein 1, is a bHLH transcription factor of the HESR (Hairy and enhancer of split-related) family of transcriptional repressors. Expression of this gene is induced by the Notch and c-Jun signal transduction pathways. As disclosed herein, HEY1 expression may correlate with resistance to endocrine therapy in ER+ cancers. That is, high expression of HEY1 may indicate that an individual with ER+ cancer may be resistant to endocrine therapy for the ER+ cancer. HEY1 may be used alone, or in combination with one or more of ALDH1, Notch, HES1 or HEY2.

HEY2 (GenBank NP036391.1; GI: 6912414)(SEQ ID NO:5) also known as Hairy/enhancer of split related with YRPW motif protein 2 and cardiovascular helix-loop-helix factor 1 (CHF1) is member of the HESR family of bHLH transcription repressors. As disclosed herein, HEY2 expression may correlate with resistance to endocrine therapy in ER+ cancers. That is, high expression of HEY2 may indicate that an individual with ER+ cancer may be resistant to endocrine therapy for the ER+ cancer. HEY2 may be used alone, or in combination with one or more of ALDH1, Notch, HES1 or HEY1.

### Subjects

Subjects to which the present methods may be applied may be any animal or human. The subject is preferably mammalian, more preferably human. The subject may be a non-human mammal, but is more preferably human. The subject may be male or female. The subject may be a patient. Therapeutic uses may be in human or animals (veterinary use). Breast cancer is more commonly (although not exclusively) associated with females, so in many cases, the subject is female.

The subject may be a patient. That is to say that the subject may be supervised by a physician or medical professional in relation to the disease or disorder to which the invention pertains. Thus, in some cases the subject has cancer, and the subject may be a cancer patient. The methods according to the present invention may be directed to cancerous conditions in female patients.

### Subject Selection

Methods disclosed herein relate to the selection of subjects. As used herein, subjects who are considered suitable for treatment are those subjects who are expected to benefit from, or respond to, the treatment.

Subjects may have, or be suspected of having, or be at risk of having cancer. Subjects may have received a diagnosis of cancer. In particular, subjects may have breast cancer. The cancer may be Estrogen Receptor positive (ER+) cancer.

Methods of determining whether an individual has an Estrogen Receptor positive cancer are well known in the art, and any of these methods may be used to determine whether a particular cancer is ER+. Tests include determining the percentage of cells in a sample that stain positive for the estrogen receptor, and Allred scoring which considers the intensity of staining in addition to the proportion of cells that are stained. Cancers with low numbers of hormone receptors may still respond to endocrine therapy.

Subjects may have, or be suspected of having, or be at risk of developing, endocrine therapy resistant ER+ breast cancer. Resistance to endocrine therapy may be intrinsic or acquired. Resistance to endocrine therapy may manifest itself as clinical progression of the cancer (i.e. the endocrine therapy does not, or does not significantly, alter the progression of the cancer). For example, increased tumor grade or increased proliferation of the cancer whilst undergoing endocrine therapy. Where the resistance is acquired, resistance may manifest as relapse or recurrence of cancer during or after completion of endocrine therapy. For example, the primary tumor may increase in size or increase in grade, or the cancer may spread to regional nodes or beyond to distant metastatic sites. The subject may initially respond to the endocrine therapy, with a subsequent decrease in the degree to which they respond.

Methods disclosed herein relate to the reversal of resistance to endocrine therapy, or the induction of sensitivity to endocrine therapy. That is, subjects with ER+ cancer, who are not responding to endocrine therapy, or who are determined will not respond to endocrine therapy, can be induced to respond to endocrine therapy by the methods described herein.

A subject who is sensitive to endocrine therapy exhibits a reduction in tumor progression in response to endocrine therapy. For example, the subject may exhibit at least a 1%, 5%, 10%, 15%, 20%, 25%, 30%, 35%, 40%, 45%, 50%, 55%, 60%, 65%, 70%, 75%, 80%, 85%, 90%, 95% or 100% reduction in tumor size, or in the number of tumors in response to endocrine therapy. In some cases, the reduction is in the size or number of primary tumors. The reduction may additionally, or alternatively, be in the size or number of metastatic tumors.

### Cancer

Methods and compositions disclosed herein are particularly relevant to breast cancer, and to estrogen positive breast cancer (ER+). However, the methods may also be relevant to other cancers that are hormone dependent, such as other types of estrogen receptor positive cancers. The cancer may be a primary cancer or a secondary cancer, including metastatic breast cancer, and metastatic estrogen receptor positive breast cancer.

A “cancer” may also comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

### Detection and Quantification

Methods disclosed herein involve the detection and/or quantification of ALDH1. Detection, as used herein, refers to measurement of ALDH1 without quantification. Methods for detection and quantification of ALDH1 nucleotides and proteins are well known in the art and will be readily appreciated by a skilled person.

Methods according to the present invention may be performed in vitro or ex vivo. The term “in vitro” is intended to encompass experiments with materials, biological substances, cells and/or tissues in laboratory conditions or in culture. “Ex vivo” refers to something present or taking place outside an organism, e.g. outside the human or animal body, which may be on tissue (e.g. whole organs) or cells taken from the organism.

The methods disclosed herein relate to the determination of protein expression. Protein expression can be measured by quantifying the amount of protein in a cell, tissue or sample, or by observing the localization of the protein within cells and tissues.

In some cases, immunoassays are used to detect the target in a sample from the subject. Immunoassays use antibodies with specific affinity for the target molecule in conjunction with a detectable molecule. In some cases, the antibody is conjugated to the detectable molecule. The detectable molecule may be referred to as a label. The detectable molecule produces a detectable signal when the antibody is bound to the target molecule.

The detectable signal may be a quantifiable signal. In some cases, an aptamer is used instead of, or together with, the antibody. Immunoassays include immunohistochemistry, ELISA, immunoblotting and flow cytometry. In certain aspects described herein, the assay is an immunohistochemistry assay. Such assays commonly use antibodies, although other target specific molecules such as aptamers or other ligands may be used.

The method may be approved for use by a regulatory agency. The method may be an FDA approved method.

### Immunohistochemistry

Immunohistochemistry (IHC) is broadly used and well established as a diagnostic test methodology particularly in oncology indications and provides highly accurate results if used under standardized conditions (Demidova, Barinov et al., 2014).

IHC refers to the process of detecting targets in cells of a tissue section by exploiting the principle of antibodies binding specifically to the target in biological tissues. IHC is widely used in the diagnosis of abnormal cells, such as those found in cancerous tumors. Visualizing an antibody-target interaction can be accomplished in a number of ways. Commonly, an antibody is conjugated to label. Alternatively, the antibody is detected by a secondary antibody, which is itself labelled. Detection of the label is thus indicative of the presence of target. IHC can be used to determine the cellular localization of a target and the amount of target present. IHC may be qualitative or semi-quantitative.

Immunohistochemistry methods are known in the art and are suitable for use as described herein. IHC methods commonly involve the fixation of a sample so that the sample is preserved from degradation. In certain aspects, a sample is formalin fixed and paraffin embedded (FFPE). In other aspects, IHC is performed on frozen samples. Prepared samples may be sectioned prior to analysis.

The sample may undergo pre-treatment, such as with Ventana CC1 (Cell Conditioning 1) solution. Where the sample is a FFPE sample, the method may involve deparaffinization of the sample.

Prepared samples are incubated with an antibody that is specific to the target. The samples may be incubated with an anti-ALDH1 antibody. The conditions and duration of incubation will depend on the particular antibody used. In some cases, the sample is incubated for between 10 minutes and 60 minutes, between 20 minutes and 45 minutes, or between 25 minutes and 35 minutes. In some cases, the sample may be incubated with the antibody for around 30 minutes, such as for 32 minutes. Incubation may occur at room temperature, or between about 20° C. and 50° C., between 30° C. and 40° C., or around 35° C., such as 37° C. Preferably the sample is incubated with the antibody for 32 minutes at 37° C.

The samples may additionally be counter-stained to facilitate analysis. For example, the sample may be stained with haematoxylin and eosin (H&E) stained.

The methods disclosed herein may be performed manually or automatically. Preferably, the methods are at least partially automated. For example, slide staining steps may be automated. Slide staining may be performed using a Ventana™ BenchMark ULTRA™. Alternatively, slide staining may be performed using a Ventana™ BenchMark XT™, Ventana™ BenchMark GX™, Dako Omnis™, Dako AutostainerLink48™, Leica™ BOND RX™, Leica™ BOND-III™ or Leica™ BOND MAX™

Following incubation of the sample with the labelled antibody, they may be analysed using a microscope.

### ELISA

In some cases, the target may be detected by ELISA (enzyme-linked immunosorbent assay). Target molecules from a sample are attached to a surface and detected using a specific antibody. The target may be attached to the surface non-specifically (via adsorption to the surface) or specifically (using a specific capture agent such as an antibody). ELISA may be used to quantify target in a sample. ELISA is particularly suited to the analysis of liquid samples, such as serum, urine or saliva.

### Immunoblotting

In some aspects, the target is detected by immunoblotting, or western blotting. In such methods, proteins in a sample are separated based on their electrical charge or size. They may be separated by an electrophoresis based method. The separated proteins are transferred to a membrane, where they are stained with an antibody that is specific to the target. The antibody is then detected, either directly by virtue of the antibody being conjugated to a detectable label, or indirectly, by adding a labelled secondary antibody.

### Flow Cytometry

Flow cytometry based biomarker detection may be used to detect cells expressing a biomarker of interest, such as ALDH1. Cells from the sample are suspended in a stream of fluid and directed past an electronic detection apparatus. The cells may be labelled with an antibody that is specific to the biomarker of interest. In particular, the cells may be labelled with a fluorescent antibody. Cells that express the biomarker of interest may be detected and quantified, based on the fluorescent signal from the label.

A type of flow cytometry useful in the methods disclosed herein is Fluorescence Activated Cell Sorting (FACS). Using FACS, cells may be separated into two or more vessels, based on the presence or absence of the fluorescent label on the cell.

### Hybridisation

Certain aspects herein relate to the detection of a nucleic acid of interest, such as a ALDH1 nucleic acid. The nucleic acid may be a genomic nucleic acid or a transcribed nucleic acid, such as an mRNA. The method may involve the generation of cDNA from a mRNA of interest.

Suitable methods for the detection of nucleic acids include a hybridization step, in which a nucleic acid of interest is complementary to, and binds to, a nucleic acid molecule with a known sequence. The nucleic acid molecule with a known sequence may be a probe or primer, and may be synthetic. It may be labelled, such as with a radioactive moiety or a colourimetric moiety. Nucleic acid detection methods may be qualitative or quantitative. Such methods may also be used to detect the location of a nucleic acid of interest within a cell, tissue or organism.

Methods for the detection of nucleic acid include PCR based methods, such as rtPCR and qPCR. Other methods include northern and Southern blotting. Such methods involve separation of fragments, such as by electrophoresis, and subsequent detection of nucleic acid by probe hybridization.

Further methods include in situ hybridization methods, such as Fluorescent in situ hybridization (FISH). FISH uses fluorescent probes that bind only those parts of the chromosome with which they show a high degree of sequence complementarity. FISH may also be used to detect RNA targets, such as mRNA. FISH may be used to detect nucleic acid in cells, circulating tumor cells and tissue samples.

### Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind ALDH1 (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

Anti-ALDH1 antibodies are known in the art, including commercially available antibodies.

### Elevated Expression or Activity

As disclosed herein, elevated ALDH1 expression may indicate that a subject is, or will become, resistant to endocrine therapy for ER+ breast cancer. As used herein, elevated expression is used interchangeably with increased expression, or high expression.

Elevated expression means an increase in the level of ALDH1 protein or nucleic acid. The expression may be elevated locally or globally, for example within a particular tissue or cell type, such as within a tumor or within bone marrow, or maybe elevated throughout the body of the patient. Elevated expression may be caused by an increase in production of that protein or nucleic acid, or by a decrease in the elimination or destruction of that protein or nucleic acid, or both.

Elevated activity may be caused by an increase in the amount of the protein or nucleic acid, or by an increase in the activity of each individual molecule. This may occur through a mutation in the gene or protein sequence, such as an activating mutation, or may be due to a post-translational change, such as aberrant protein phosphorylation.

In some cases, the expression of ALDH1 is significantly upregulated in the subject or sample, relative to a control.

Overexpression or increased activity of ALDH1 relative to a control is indicative of a poor prognosis and poor survival. Very high overexpression or very high activity of ALDH1 is indicative of a resistance to endocrine therapy.

In some cases, expression or activity of 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 750% or 1000% or a higher percentage more than the expression or activity in the control is indicative of resistance to endocrine therapy.

In some cases, expression or activity of 1.5 times, 2 times, 3 times, 4 times, 5 times, 6 times, 7 times, 8 times, 9 times, 10 times, 15 times, 20 times, 25 times, 30 times, 35 times, 40 times, 45 times, 50 times, 100 times, or more times more than the expression or activity in the control is indicative of resistance to endocrine therapy.

### Control

In some cases, the method involves comparing ALDH1 in a sample from a patient with ALDH1 in one or more control samples.

Suitable control samples and subjects will be appreciated by those of skill in the art. In particularly preferred aspects disclosed herein, the control is a sample previously obtained from the same patient, at an earlier point in time. For example, in a sample obtained from a subject before undergoing endocrine therapy, or before undergoing the particular endocrine therapy to which they have become resistant. In some cases, the control is obtained from an individual who is known to have a cancer that is responsive to endocrine therapy. For example, an individual that is known to have endocrine therapy responsive ER+ breast cancer.

The comparison may not require the analysis of the control sample to be simultaneously or sequentially performed with the analysis of the sample from the patient. Instead, the comparison may be made with results previously obtained from a control sample, such as results stored in a database.

The control sample may be a sample obtained from the patient prior to the onset of cancer, or prior to the observation of symptoms associated with cancer.

The control sample may be a sample obtained from another individual, such as an individual who does not have cancer. The individual may be matched to the patient according to one or more characteristics, for example, sex, age, medical history, ethnicity, weight or expression of a particular marker. The control sample may have been obtained from the bodily location, or be of the same tissue or sample type as the sample obtained from the patient.

The control sample may be a collection of samples, thereby providing a representative value across a number of different individuals or tissues.

### Hybridization

Certain methods described herein involve nucleotide sequences that are capable of hybridizing selectively to any of the ALDH1 sequences described herein or to the complement of any of the above. Nucleotide sequences may be at least 1 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridizing to the nucleotide sequences presented herein, or to their complement, will be generally at least 70%, such as at least 80 or 90% and such as at least 95% or 98% homologous to the corresponding nucleotide sequences presented herein over a region of at least 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screened. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

The polynucleotides described here may be used to produce a primer, e.g. a PCR primer, a primer for an alternative amplification reaction, a probe e.g. labeled with a revealing label by conventional means using radioactive or non-radioactive labels, or the polynucleotides may be cloned into vectors. Such primers, probes and other fragments will be at least 15, such as at least 20, for example at least 25, 30 or 40 nucleotides in length, and are also encompassed by the term polynucleotides as used herein. Fragments may be less than 500, 200, 100, 50 or 20 nucleotides in length.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

### Samples

The methods of the invention may involve determining the level of a biomarker in a sample. Methods described herein may be performed on a sample that has been obtained from a patient. Such methods may thus be performed ex vivo. They may be performed in vitro.

A sample may be taken from any tissue or bodily fluid. In some cases the sample is a tumor tissue sample, such as a tumor biopsy.

In other cases the comprises or is derived from: a quantity of blood; a quantity of serum derived from the individual's blood which may comprise the fluid portion of the blood obtained after removal of the fibrin clot and blood cells, a quantity of plasma, or cells derived from a blood sample.

In preferred arrangements the sample is taken from a bodily fluid, more preferably one that circulates through the body. Accordingly, the sample may be a blood sample or lymph sample.

In a particularly preferred arrangement the sample is a blood sample or blood-derived sample. The blood derived sample may be a selected fraction of a patient's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present invention may involve detection of a BORIS polypeptide or nucleic acid in the blood, in white blood cells, peripheral blood mononuclear cells, granulocytes and/or red blood cells.

A selected serum fraction may comprise the fluid portion of the blood obtained after removal of the fibrin clot and blood cells. In another arrangement the sample may be taken from blood precursor tissues, such as blood cell progenitor cells, bone marrow or spleen.

Alternatively the sample may comprise or may be derived from a tissue sample, biopsy or isolated cells from said individual.

### Prognosis

Prognosis, prognosing and prognose refer to estimating the risk of future outcomes in an individual based on their clinical and non-clinical characteristics. In particular, a method of determining the prognosis as used herein refers to the prediction of the outcome of, or future course of, an individual's or subject's cancer and, in particular, whether the subject is likely to respond to endocrine therapy. Prognosis includes the prediction of patient's survival. Prognosis may be useful for determining an appropriate therapeutic treatment. Prognostic testing may be undertaken with (e.g. at the same time as) the diagnosis of a previously undiagnosed cancerous condition, or may relate to an existing (previously diagnosed) condition.

The method of prognosis may be an in vitro method performed on the patient sample, or following processing of the patient sample. Once the sample is collected, the patient is not required to be present for the in vitro method of prognosis to be performed and therefore the method may be one which is not practiced on the human or animal body. Additional prognostic indicators may include the analysis of expression levels or activity of proteins or nucleic acids, such as oncogenes or known prognostic marker genes, expression of known mutant proteins, nucleic acids or genes, or other factors such as the age, sex, general health, symptoms, signs, test results or medical history of the patient.

Clinical and non-clinical prognostic indicators will be readily appreciated to those of skill in the relevant art.

The prognosis may be for a sample or patient that has a normal karyotype. In some cases, the sample or patient may exhibit an abnormal karyotype, such as an abnormal number or structure of chromosomes or other cytogenetic complication.

The sample may be from a patient who has already been treated with an anti-cancer therapy, such as chemotherapy, radiotherapy or, in particular, with hormone treatment. In some cases, the cancer will not have responded to the anti-cancer therapy.

Prognosis may be used to predict the disease free survival time of an individual, progression-free survival time, disease specific survival time, survival rate, or survival time. In particularly preferred aspects, prognosis is used to predict the likelihood that a subject will respond to endocrine therapy.

Survival rate (also known as overall survival) is the percentage of people who are alive for a given period of time after prognosis. For example, the percentage of people who are alive 1 month, 3 months, 6 months, 12 months, 18 months, 24 months, 3 years, 4, years, 5 years, 10 years or longer, after the prognosis is made. Survival rate may be a percentage likelihood that the patient will be alive in a particular period of time, for example, a 5%, 10%, 15%, 20%, 25%, 30%, 35%, 40%, 45%, 50%, 55%, 60%, 65%, 70%, 75%, 80%, 85%, 90%, 90% or 100% likelihood that a patient will be alive in 1 month, 3 months, 6 months, 12 months, 18 months, 24 months, 3 years, 4, years, 5 years, 10 years or longer.

Survival time is the remaining duration of life of the patient. For example an estimated time of 1 month, 3 months, 6 months, 12 months, 18 months, 24 months, 3 years, 4, years, 5 years, 10 years or longer time alive following the prognosis.

Disease free survival time (DFS) is the length of time after a primary treatment for a cancer ends that the patient survives without signs or symptoms of that cancer.

Progression-free survival time is the length of time during which the disease does not get worse.

The survival time may be a disease-specific survival time, which is the percentage of people who are alive for a given period of time after prognosis, treating deaths from other causes than the cancer as withdrawals from the population that don't lower survival, and thus comparable to patients who are not observed any longer (e.g. due to reaching the end of the study period).

A poor prognosis is a prediction that a disease, such as cancer, will not respond to endocrine therapy, and may recur or worsen. It may be an indication that the patient will die from the disease or cancer. A poor prognosis may be indicative of a more aggressive cancer or a late stage or metastatic cancer. One or more of a short survival time, short survival rate, short disease free survival time, short progression-free survival time, short disease specific survival time are indicative of a poor prognosis.

### Diagnosis

Diagnosis refers to the identification of a disease, such as cancer or a specific type of cancer. Methods described herein may be used to diagnose endocrine therapy resistant cancers, particularly endocrine therapy resistant ER+ breast cancer.

Detection in a sample of ALDH1 polypeptides or nucleic acids in accordance with the methods of the present invention may be used for the purpose of diagnosis of a resistance to endocrine therapy in a subject, diagnosis of a predisposition to resistance to endocrine therapy in a subject or for determining a prognosis (prognosticating) the likely success of endocrine therapy. The diagnosis or prognosis may relate to an existing (previously diagnosed) cancerous condition, which may be benign or malignant, may relate to a suspected cancerous condition or may relate to the screening for cancerous conditions in the patient (which may be previously undiagnosed).

Other diagnostic tests may be used in conjunction with those described here to enhance the accuracy of diagnosis or prognosis of a cancerous condition or to confirm a result obtained by using the tests described here.

The method of diagnosis may be an in vitro method performed on the subject's sample, or following processing of the subject's sample. Once the sample is collected, the subject is not required to be present for the in vitro method of diagnosis to be performed and therefore the method may be one which is not practiced on the human or animal body.

Other diagnostic tests may be used in conjunction with those described here to enhance the accuracy of the diagnosis or prognosis or to confirm a result obtained by using the tests described here.

### Compositions

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

A composition may be administered alone or in combination with other treatments, either simultaneously or sequentially dependent upon the condition to be treated.

### Detection

Detection in a sample of polypeptides or nucleic acids in accordance with the methods of the present invention may be used for the purpose of diagnosis of a cancerous condition in the subject, diagnosis of a predisposition to a cancerous condition or for providing a prognosis (prognosticating) of a cancerous condition. The diagnosis or prognosis may relate to an existing (previously diagnosed) cancerous condition, which may be benign or malignant, may relate to a suspected cancerous condition or may relate to the screening for cancerous conditions in the subject (which may be previously undiagnosed). Other diagnostic tests may be used in conjunction with those described here to enhance the accuracy of diagnosis or prognosis of a cancerous condition or to confirm a result obtained by using the tests described here.

The method of diagnosis may be an in vitro method performed on the subject sample, or following processing of the subject sample. Once the sample is collected, the subject is not required to be present for the in vitro method of diagnosis to be performed and therefore the method may be one which is not practised on the human or animal body.

Other diagnostic tests may be used in conjunction with those described here to enhance the accuracy of the diagnosis or prognosis or to confirm a result obtained by using the tests described here.

### Therapy

The methods of the invention include methods of chemotherapy and endocrine therapy. Chemotherapy refers to treatment of a tumor with a drug or with ionising radiation (e.g. radiotherapy using X-rays or y-rays). Endocrine therapy refers to treatments that target the hormone receptor by blocking receptor binding with an antagonist or by depriving the tumor of hormone.

Endocrine therapy is also known as hormone therapy. Endocrine therapy lowers the level of hormone in a subject, or lowers the effect of the hormone. Endocrine therapy may be used in conjunction with chemotherapy. That is, the endocrine therapy is administered simultaneously or sequentially with chemotherapy. In particularly preferred cases, endocrine therapy is administered in conjunction with an inhibitor of Notch signaling, particularly Notch4 signaling. The endocrine therapy may be administered in conjunction with a gamma secretase inhibitor (GSI). The different therapies may be administered simultaneously or sequentially. The endocrine therapy may be estrogen therapy.

For example, methods of the invention are particularly related to the naturally occurring hormone estrogen. Estrogen can stimulate the growth of some cancer cells, particularly breast cancer cells. Endocrine therapy for breast cancer may lower the level of estrogen in the body, or block its effects. Such therapy may be useful for cancers that are responsive to estrogen. In particular, such therapy may be useful for cancers that contain cells that have estrogen receptors. That is, estrogen receptor positive (ER+) cancers.

Endocrine therapy is the most effective treatment for ER+ metastatic breast cancer, but its effectiveness is limited by high rates of de novo resistance, and resistance acquired during treatment. Endocrine therapy relating to estrogen includes agents that interfere with estrogens ability to stimulate the growth of breast cancer cells. Selective Estrogen Receptor Modulators (SERMs) bind to estrogen receptors to prevent estrogen binding. Examples include tamoxifen (Nolvadex®), raloxifene (Evista®) and toremifene (Fareston®). Selective Estrogen Receptor Downregulators (SERDs) work to block the effect of estrogen on cells. Examples include fulvestrant (Faslodex®).

According to the invention, certain methods may involve the administration of chemotherapeutic treatments in addition to endocrine therapy. In particular, the invention relates to the administration of agents that inhibit NOTCH4 Signaling (INSs).

Inhibitors of NOTCH signaling include gamma secretase inhibitors, antibodies to Notch receptors, antibodies to Notch ligands and decoys of Notch ligands. Inhibitors of Notch signaling may reduce or ablate Notch signaling. Methods for determining the level of Notch signaling are known in the art (see, for example, Zacharioudaki and Bray (2014) Methods 68(1) 173-182), and may be used to determine whether or not an agent is an inhibitor of Notch signaling. In certain embodiments, the inhibitors are inhibitors of Notch4 signaling.

One class of NOTCH signal inhibitors useful in the methods disclosed herein include the Gamma Secretase Inhibitors (GSIs). There are more than 1000 GSIs synthesized to date, divisible into three separate classes: peptide isosteres, azepines, and sulphonamides. GSIs block NOTCH activity by preventing its cleavage at the cell surface.

GSIs relevant to the methods disclosed herein include RO4929097 (Roche), GSI MK-752 (Merck), PF03084014 (Pfizer) and BMS-906024 (Bristol-Myers Squibb).

Also useful in the methods disclosed herein are antibodies to notch ligands, such as antibodies that bind to JAGGED1 (GenBank: AAC52020.1 GI: 1695274), (SEQ ID NO:6) JAGGED 2 (GenBank: AAB84215.1 GI: 2605945) (SEQ ID NO:7) and DLL4 (GenBank: AAQ89253.1; GI:37182906)(SEQ ID NO:8). An example of a DLL4 inhibitor suitable for use in the methods of the invention is MED10639 (MedImmune).

Also useful in the methods disclosed herein are molecular decoys. Molecular decoys selectively block the binding of a notch receptor to its, ligand, thereby modulating cell signaling. For example, molecular decoys may mimic the binding of a ligand to its receptor, without triggering downstream signaling events, or may sterically hinder binding of the receptor and ligand by blocking the ligand binding site. Molecular decoys include small molecules and peptides, including short peptide fragments of the receptor or ligand. For example, a peptide fragment comprising one or more regions of a DLL or JAG class ligand, such as those disclosed in Kangsamaksin et al., (Cancer Discovery 2015: 5(2)182-197).

Antibodies and fragments of antibodies that bind to a Notch receptor or a ligand are also useful in the methods disclosed herein. Antibodies and antibody fragments are well known in the art, and are discussed above. Suitable antibodies include OMP-59R5 (tarextumab, Oncomed), which is a fully human monoclonal antibody that targets Notch 2/3 receptors, MEDI0639 (MedImmune) and OMP-21M18 (OncoMed, humanized IgG2) monoclonal antibodies that target DLL4.

The subject may be administered other chemotherapeutic agents. In particular, where the subject is determined to have, or be at risk of having, an advanced or invasive cancer, or a cancer that is unlikely to respond to endocrine therapy, therapies other than endocrine therapy may be used in addition to, or as an alternative to, the endocrine therapy.

In preferred embodiments chemotherapy refers to treatment with a drug. The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the tumor.

The chemotherapy may be administered by one or more routes of administration, e.g. parenteral, intravenous injection, oral, or intratumoural. Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

The chemotherapy may be administered according to a treatment regime. The treatment regime may be a pre-determined timetable, plan, scheme or schedule of chemotherapy administration which may be prepared by a physician or medical practitioner and may be tailored to suit the subject requiring treatment.

The treatment regime may indicate one or more of: the type of chemotherapy to administer to the subject; the dose of each drug or radiation; the time interval between administrations; the length of each treatment; the number and nature of any treatment holidays, if any etc. For a co-therapy a single treatment regime may be provided which indicates how each drug is to be administered.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present invention, and for use in accordance with the present invention, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringers Injection, Lactated Ringers Injection. Preservatives, stabilisers, buffers, antioxidants and/or other additives may be included, as required.

### Dosage

It will be appreciated by one of skill in the art that appropriate dosages of the inhibitor of notch signaling, such as a gamma secretase inhibitor, and the endocrine therapy can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects. In some cases, the dosage of endocrine therapy may be reduced compared to established norms of dosage, when administered in conjunction with an inhibitor of Notch signaling. The dosage of endocrine therapy may be reduced as compared to the dosage previously administered to the subject, when the inhibitor of Notch signaling is added to the subjects therapy regimen.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### EXAMPLES

### Example 1

### Anti-Estrogen Resistance in Human Breast Tumors is Driven by NOTCH4-Dependent Cancer Stem Cell Activity

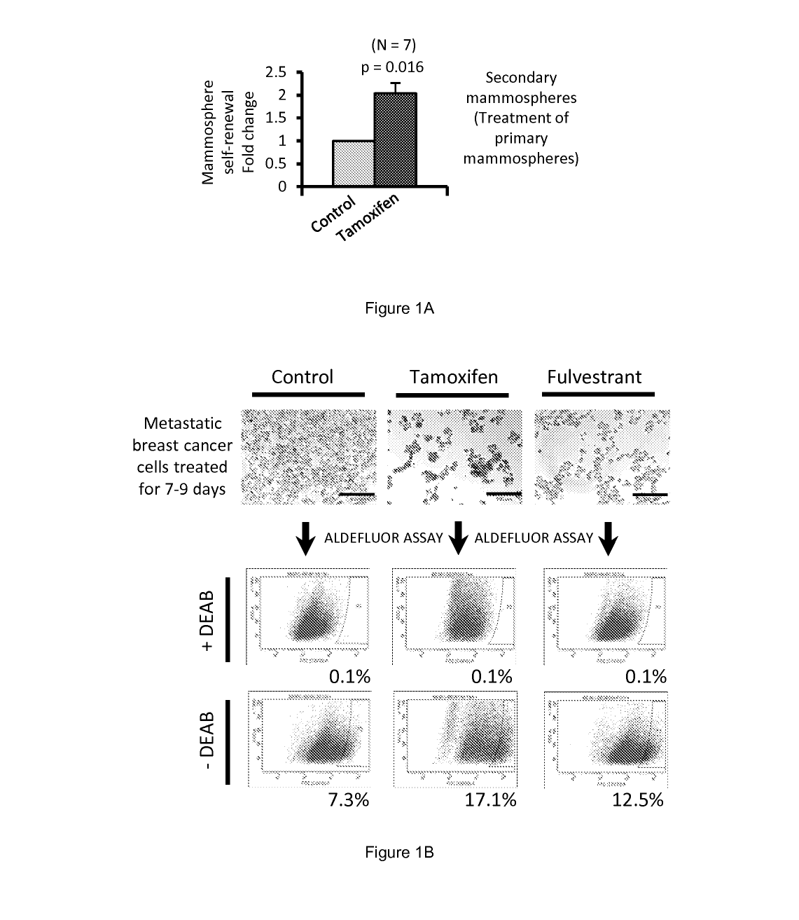

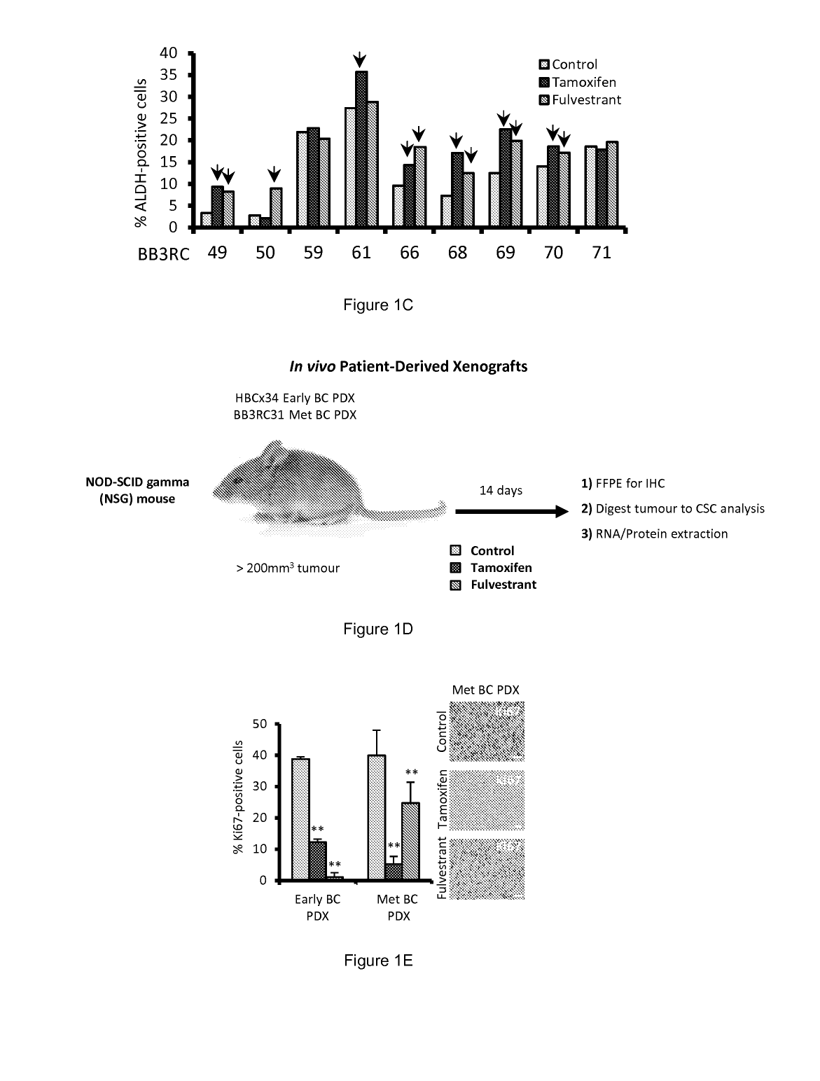

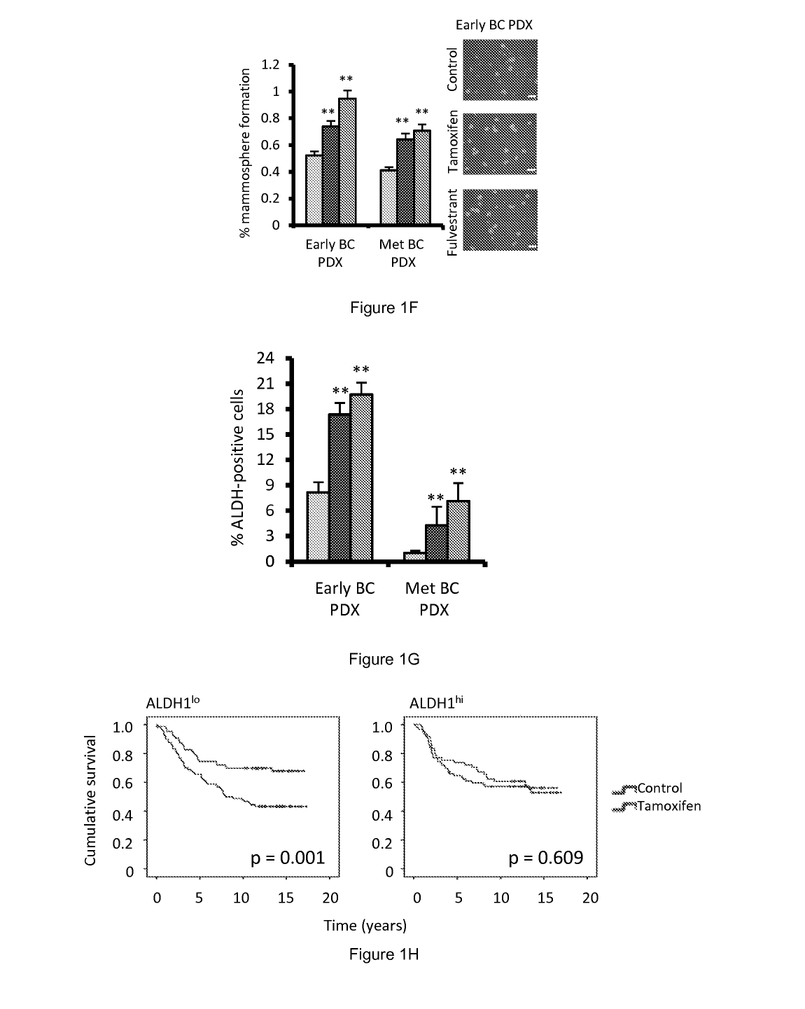

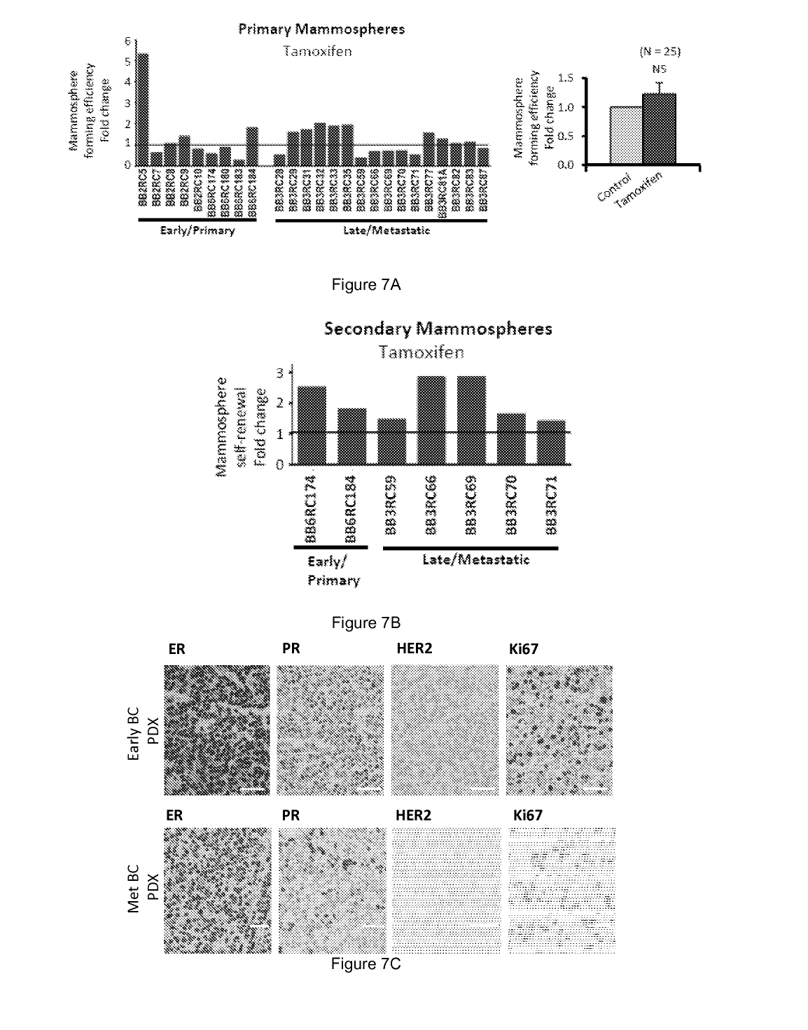

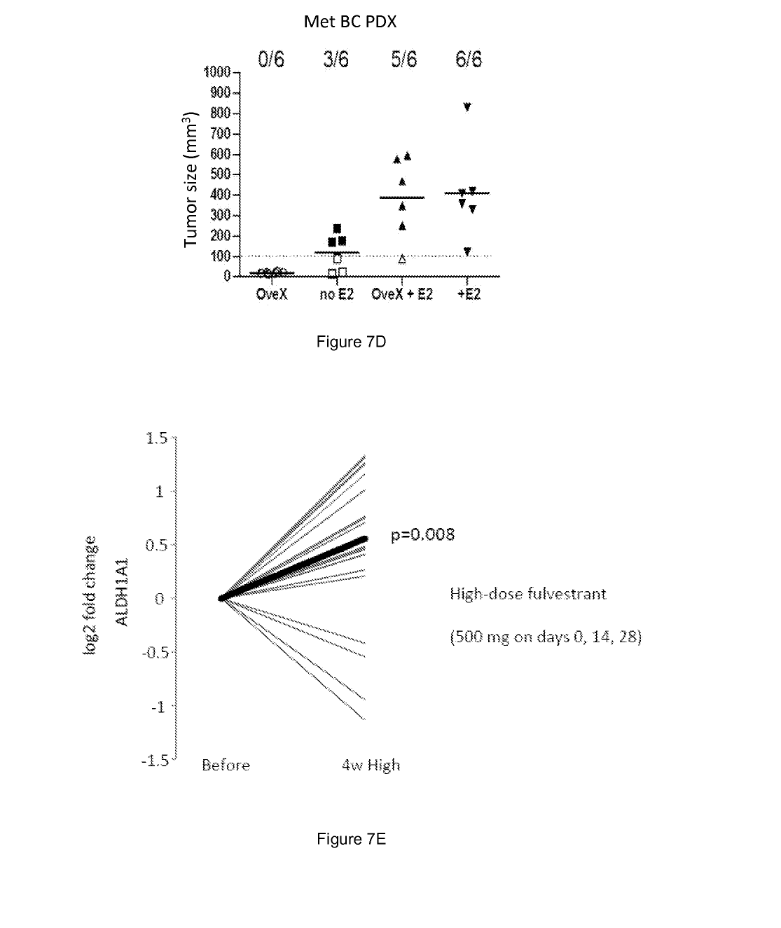

Breast cancer stem cell (BCSC) activity is enriched by tamoxifen and fulvestrant BCSCs are likely uniquely capable of resisting anticancer agents, surviving for long periods in a near quiescent state and eventually producing recurrence and metastases. BCSCs, measured by CD44+CD24^{low} expression and mammosphere formation efficiency (MFE), have been shown to be enriched by chemo- and radio-therapies (Li et al., 2008; Phillips et al., 2006). The role of BCSCs in endocrine resistance has been less studied but it has been reported that cancer stem cell-like gene expression is found in patient breast tumors after short term presurgical trials of endocrine treatment with tamoxifen (Kabos et al., 2011). We therefore tested the effect of the anti-estrogen tamoxifen on mammosphere-forming activity of patient-derived ER+ tumour cells and confirmed that tamoxifen increases mammosphere self-renewal by about two-fold (FIGS. 1A, 7A and 7B). Next, we investigated ALDH activity, another functional assay for CSCs, in samples from patients with advanced breast cancer treated with either tamoxifen or a second anti-estrogen drug, fulvestrant. Seven out of nine patient samples treated with tamoxifen or fulvestrant showed a significant increase in ALDH enzymatic activity, but one only to tamoxifen and one only to fulvestrant (FIGS. 1B and 1C). These data suggest that endocrine therapies such as tamoxifen or fulvestrant, even when given for a period of a few days, enrich for stem cell activity. Consistent with the findings, our analysis of microarray data from a recent publication reveals that expression of the ALDH1A1 gene, which encodes the ALDH enzyme, is significantly increased in breast tumors following short-term administration of fulvestrant to patients (FIG. 7C, Patani et al., 2014).

To model the short-term clinical treatment used above, we tested the in vivo impact of endocrine therapies on stem cell activity in ER+ BC using patient-derived xenografts (PDX) grown sub-cutaneously in mice. We used an early (treatment naïve) and a metastatic ER+ PDX tumor that both maintain biological characteristics (such as the expression of ER and estrogen dependence) of the patient primary tumor from which they were derived (FIGS. 7C and 7D). The estrogen dependence of the HBC×34 PDX model (Early BC) has been previously reported (Cottu et al., 2012) and we demonstrated estrogen dependence of the novel BB3RC31 PDX model (Met BC) by ovariectomy and/or estrogen supplementation in tumor growth assays (FIG. 7D). Using a 14 day in vivo ‘window’ treatment (FIG. 1D), we observed that, as previously observed in patients (Dowsett et al., 2011), both tamoxifen and fulvestrant treatment decrease the proliferation marker Ki67 (FIG. 1E) indicating that cell division is inhibited by anti-estrogen therapy. However, there is a paradoxical increase in BCSC activity measured by MFE and ALDH enzymatic activity (FIGS. 1F and 1G). These results suggest a potentially novel mechanism for endocrine resistance driven by enrichment for a stem cell phenotype rather than genetic selection in response to short-term exposure to anti-estrogens.

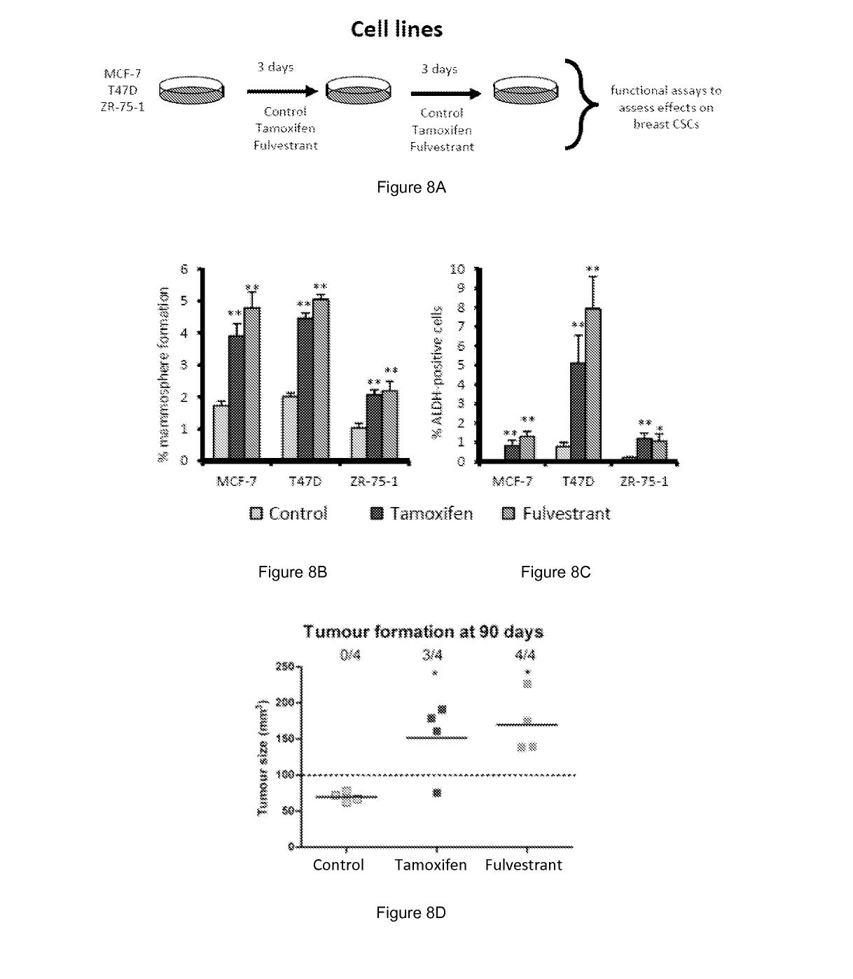

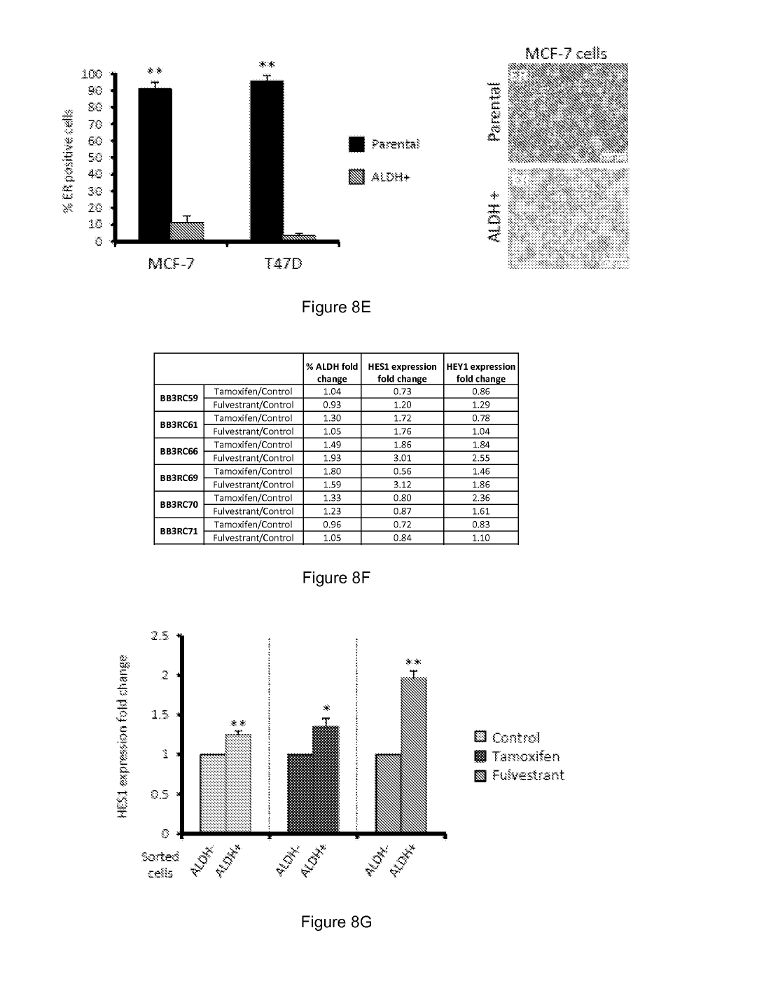

Confirming this, we find that short term in vitro pre-treatment (6 days) of ER+ cell lines (MCF-7, T47D and ZR-75-1) with tamoxifen or fulvestrant also enriched for mammosphere-forming activity, ALDH-positive cells and in vivo tumor initiation capacity (FIGS. 8A, 8B, 8C and 8D). Thus short-term anti-estrogen treatment selects for a population of cells that form mammosphere colonies in vitro and tumors in vivo. The mechanism for this stem cell enrichment by anti-estrogens may be partly explained by our data demonstrating that more than 90% of sorted ALDH+ cells are ER-negative (FIG. 8E) suggesting that anti-estrogens cannot directly target ALDH+ cells. An ALDH+ER-BCSC from which ER+ daughter cells arise in ER+ tumors would be analogous to the pattern of ER expression in the cellular hierarchy of the normal mammary gland (Honeth et al., 2014) and is consistent with previous reports of ER− BCSC-enriched populations in ER+ BC (Harrison et al., 2013).

Based on these findings, we hypothesised that BCSC frequency would predict for response to tamoxifen treatment. We analysed ALDH1 in 322 ER+ BC samples taken prior to a randomised trial of tamoxifen versus no systemic treatment. The percentage of breast tumor epithelial cells positive for ALDH1 dichotomised at the median value predicted benefit from tamoxifen such that improvement in survival (ie. a response to treatment) was only seen in women with low epithelial ALDH1 expression (FIG. 1H; Table 93). These data, from a prospective randomised trial, establish for the first time that BC stem cell frequency predicts response to tamoxifen treatment, suggesting stem cell numbers are responsible for de novo endocrine resistance.

Thus, we find not only that BCSC activity is enriched by both short-term in vitro and in vivo endocrine treatment, but that stem cell activity in tumours before endocrine treatment predicts sensitivity, establishing a novel mechanism of both de novo and acquired endocrine resistance.

### Tamoxifen or Fulvestrant Treatments Up-Regulate Notch Target Genes

We previously reported Notch signaling to be essential for BCSC survival and self-renewal (Harrison et al., 2010). We analysed the patient-derived BC cells that were treated with tamoxifen and fulvestrant in FIGS. 1B and 1C and found that increased numbers of ALDH-positive cells were strongly correlated to increased expression of Notch target genes (HEY1 and HES1) (FIGS. 2A and 8F). In addition, the BC PDX tumors treated in vivo with 2 weeks' tamoxifen or fulvestrant (FIG. 1D) showed increased HEY1 and HES1 expression (FIG. 2B), supporting an increased role for the Notch signaling pathway after endocrine therapies.

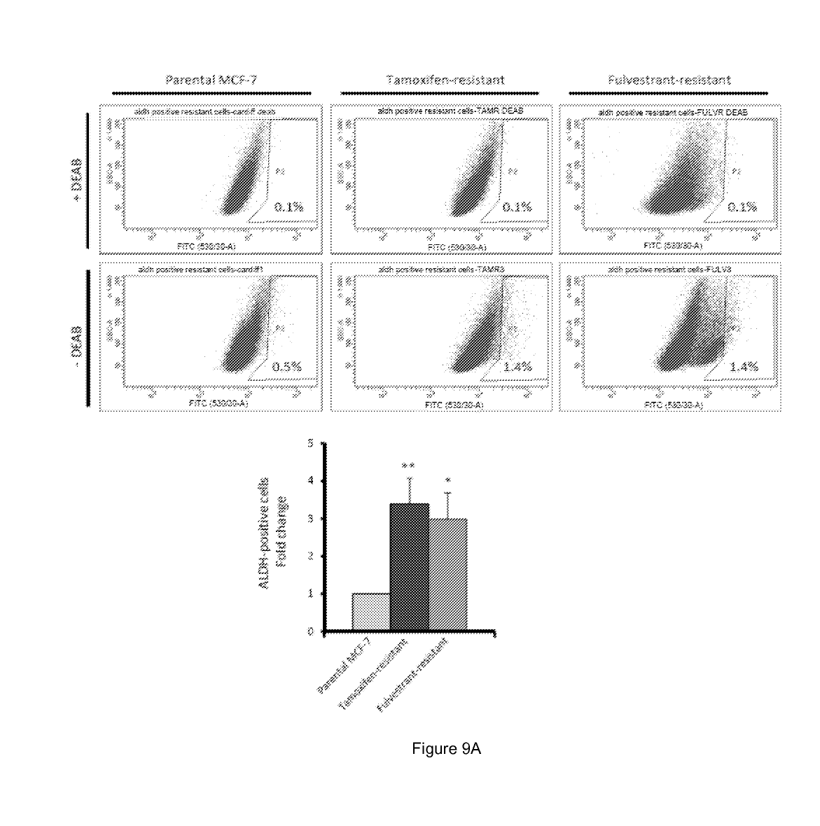

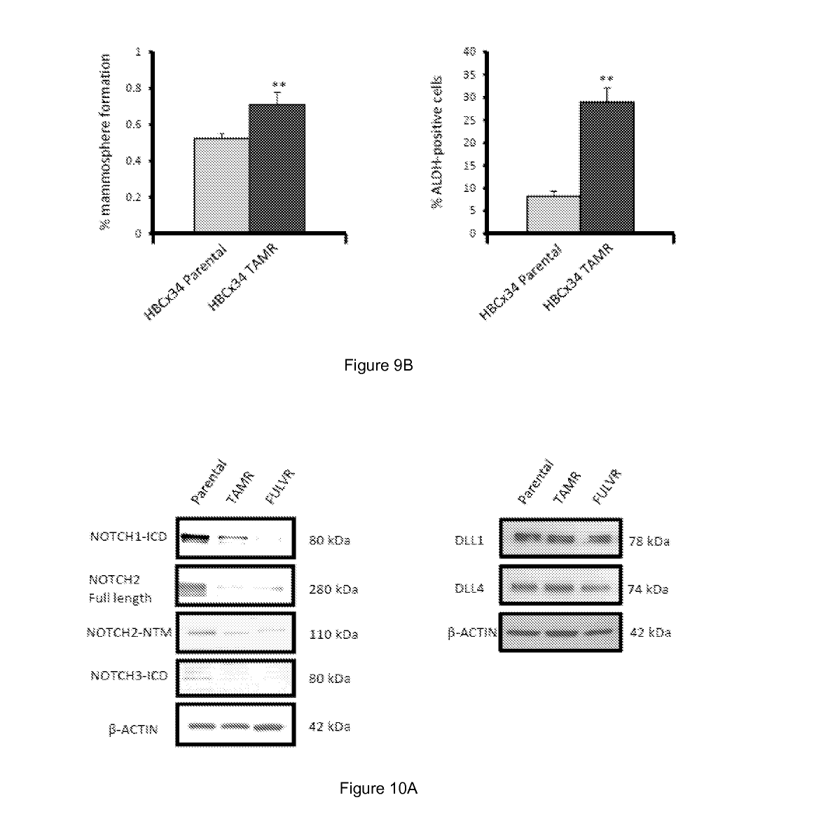

Next, we confirmed the relevance of Notch signaling in acquired endocrine resistance using ER+ cell lines (MCF-7, T47D and ZR-75-1) in vitro. Treatment with tamoxifen or fulvestrant for 6 days preferentially increased expression of HEY1 and HES1 (FIG. 2C) in the ALDH+ subpopulation (FIG. 8G). Similarly, in acquired tamoxifen (TAMR) or fulvestrant (FULVR) resistant MCF-7 models (Knowlden et al., 2003; McClelland et al., 2001), we found up-regulation of Notch target genes as well as increased Notch transcriptional activity (CBF1-luciferase reporter assay) (FIG. 2D) in addition to an increased percentage of ALDH+ cells (FIG. 9A).

Our in vitro and in vivo results indicate that tamoxifen and fulvestrant expand ALDH+ population and Notch activity. That ALDH activity and Notch genes are directly correlated is in agreement with recent reports indicating ALDH+ BCSCs are dependent upon Notch signaling and that Notch regulates ALDH enzyme activity in BC cells through SIRT2-dependent deacetylation (Hirata et al., 2014, Zhao et al., 2014).

We also analysed Notch signalling in tamoxifen and fulvestrant resistant MCF-7 cells (Dr Julia Gee, Cardiff) as an in vitro model and anti-estrogen drug resistance. We used a reporter construct comprising luciferase under the control of Notch regulated transcription factor CBF1. As shown in FIG. 2D, resistant cells exhibited increased activation of Notch signalling.

### JAG1 and NOTCH4 Receptor Signaling Drives Endocrine Resistance

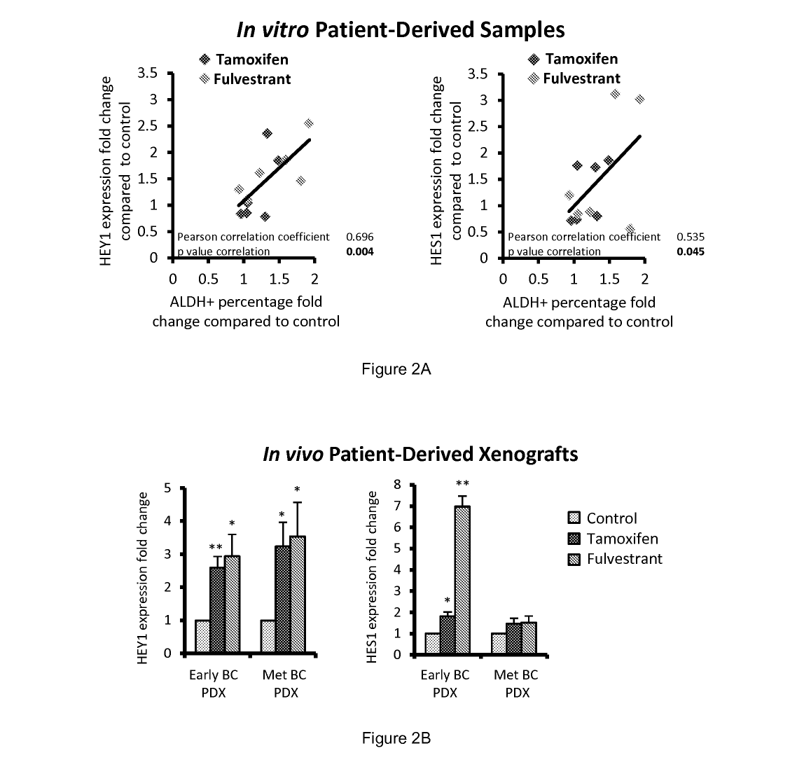

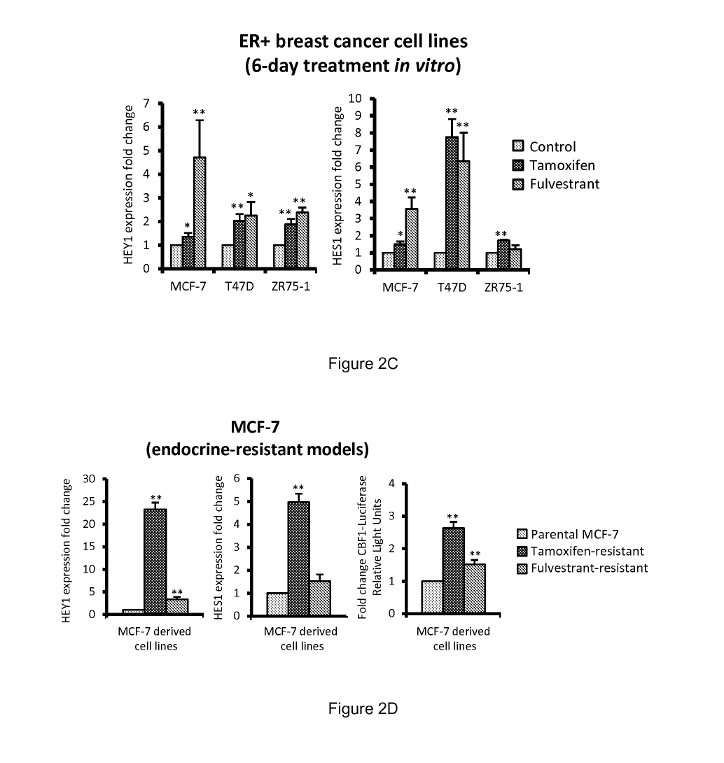

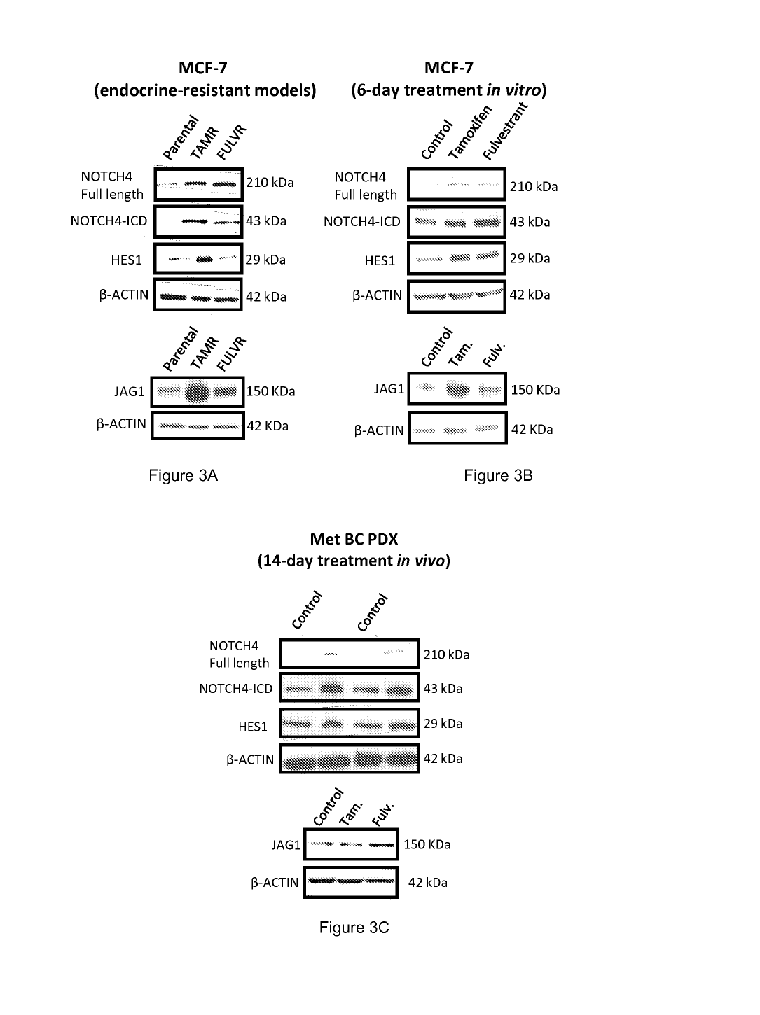

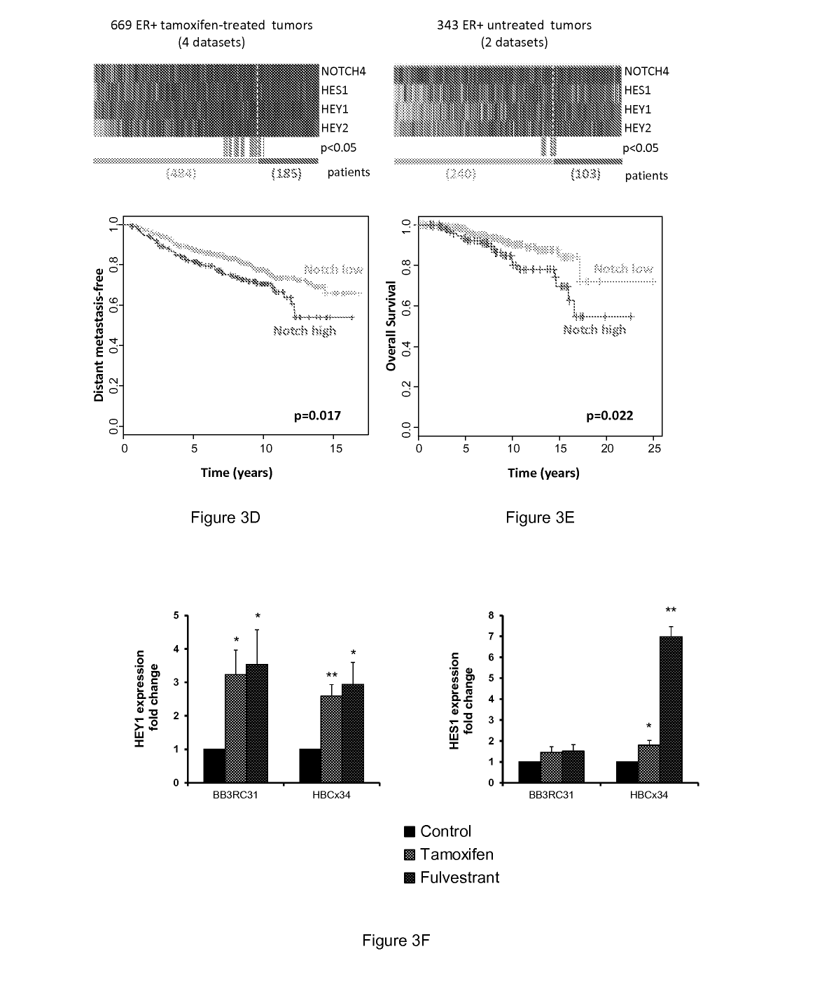

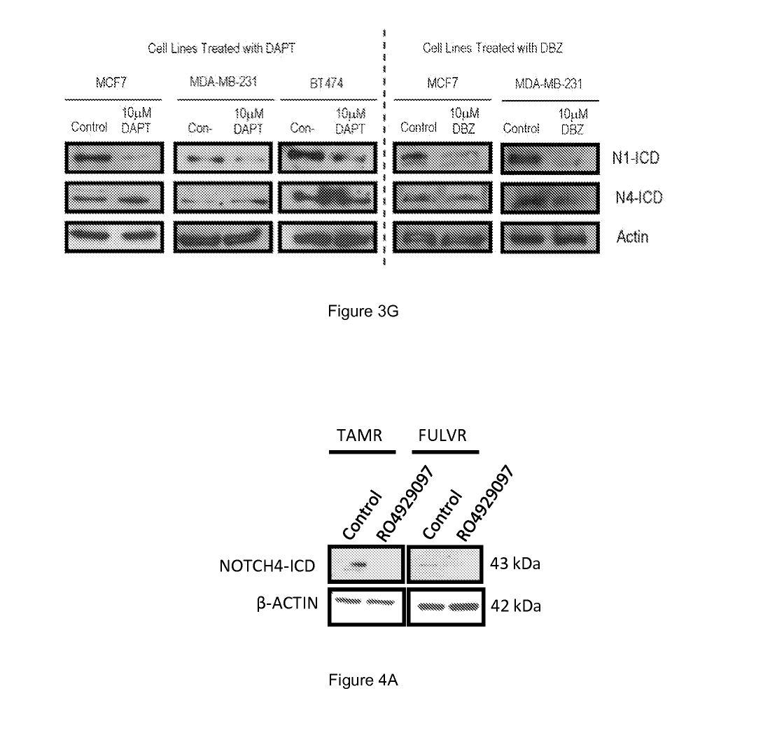

In order to investigate which of the four Notch receptors and five Notch ligands were responsible for Notch activation in endocrine-resistant models, we assessed expression levels in parental, TAMR and FULVR cell lines, which have acquired resistance after long-term tamoxifen or fulvestrant treatment. Both full length NOTCH4 and NOTCH4-ICD were up-regulated (FIG. 3A) whereas NOTCH1-3 were down-regulated (FIG. 10A) in the resistant vs. parental cell lines. We found the Notch ligand JAG1 to be highly expressed in both resistant models (FIG. 3A) while expression of the other four ligands was either unchanged (DLL1, DLL4) or absent (JAG2, DLL3) (FIG. 10A). TAMR cells displayed higher expression levels of NOTCH4-ICD, JAG1 and HES1 compared to FULVR cells, suggesting a greater degree of Notch signaling. NOTCH4-ICD and JAG1 were also up-regulated after short term treatment with tamoxifen or fulvestrant of MCF-7 cells in vitro and after a 14 day window treatment of PDXs in vivo, suggesting that activation of Notch signaling (demonstrated by increased HES1 expression) is an early event in the acquisition of endocrine resistance (FIGS. 3B and 3C).

Overall, these results indicate JAG1 ligand and cleavage of NOTCH4 intracellular domain may be responsible for Notch signaling activation after endocrine treatment. This result is in agreement with recent reports that NOTCH4 expression is increased in two independent tamoxifen-resistant cell lines and that it plays a key role in the maintenance of tamoxifen resistance in cell lines (Yun et al., 2013; Lombardo et al., 2014). The previously reported role for NOTCH4 in regulating BCSC activity (Harrison et al., 2010) adds weight to our hypothesis that anti-estrogen resistance in human breast tumors is being driven by NOTCH4-dependent BCSCs. Based on the observations above, we hypothesised that a NOTCH4/HES/HEY gene signature would also predict for response to tamoxifen treatment. In gene expression data from 669 pre-treatment tumors from four published Affymetrix microarray datasets of ER+ patients who subsequently received adjuvant tamoxifen therapy, we found NOTCH4, HES1, HEY1 and HEY2 to be co-expressed in some tumors, as demonstrated in the heatmap ordered from left to right by the sum of the four genes (FIG. 3D). Significantly, elevated expression of these Notch genes before treatment was significantly associated with distant metastasis (FIG. 3D) and with reduced overall survival in an independent cohort of 343 untreated ER+ patients (FIG. 3E). Thus, NOTCH4 gene expression and activity in tumours before treatment with endocrine therapy predicts sensitivity to treatment, indicating that this signaling pathway predicts de novo as well as acquired endocrine resistance.

Gamma secretase inhibitors DAPT and DBZ inhibited Notch1 activity in cells, but no Notch4, when we tested MCF7, MDA-MB-231 and BT474 cells. FIG. 3F.

GSI RO4929097 abrogates tamoxifen and fulvestrant stimulated CSC activity

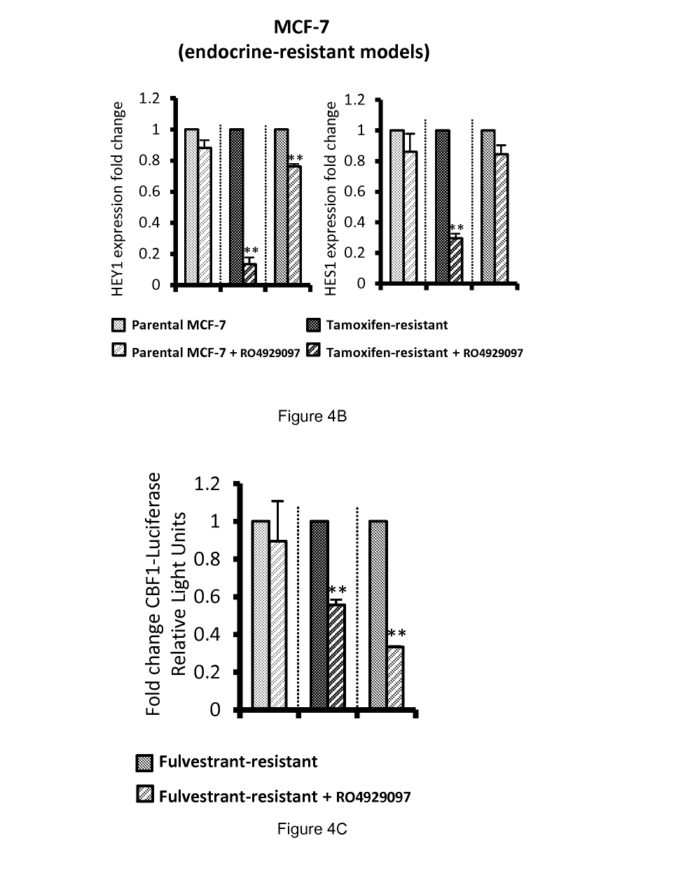

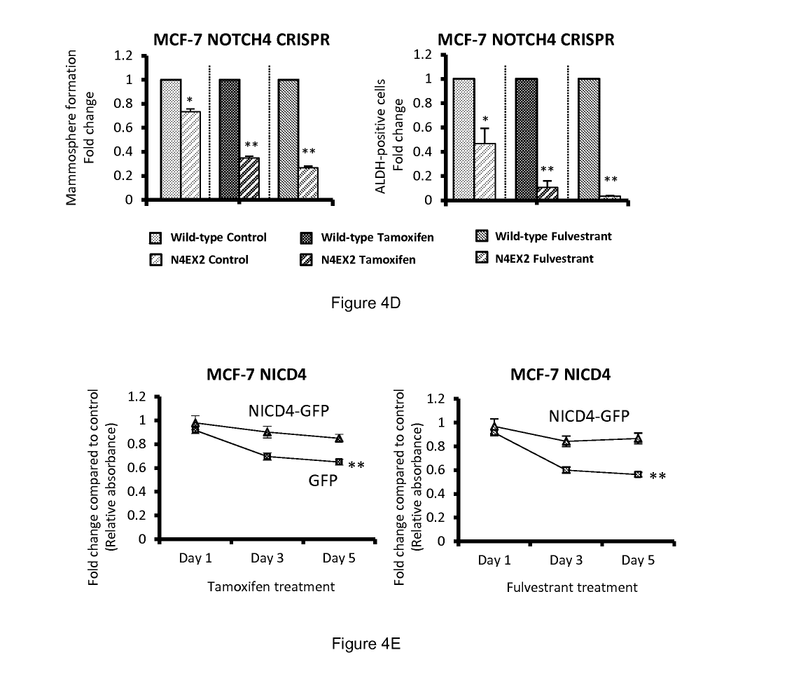

In order to inhibit NOTCH4 signaling, we used the gamma-secretase inhibitor (GSI) RO4929097, which we found to be effective in reducing levels of the active NOTCH4 intracellular domain in endocrine-resistant models (FIG. 4A). RO4929097 inhibited HEY1 and HES1 expression as well as CBF1-Notch transcriptional activity in TAMR and FULVR cell lines, but not in parental MCF-7 cells (FIGS. 4B and 4C). To further elucidate the role of NOTCH4 activity in endocrine resistance and the stem cell phenotype, we down-regulated or over-expressed NOTCH4-ICD in MCF-7 cells. Knockdown of NOTCH4 led to a significant inhibition of MFE and ALDH-positive cells, especially after tamoxifen and fulvestrant treatments (FIG. 4D). In contrast, overexpression of NOTCH4-ICD conferred tamoxifen and fulvestrant resistance in parental MCF-7 cells (FIG. 4E). Overall, these results suggest that the effects of the GSI inhibitor RO4929097 are predominantly through inhibition of NOTCH4 receptor cleavage and transcriptional activity.

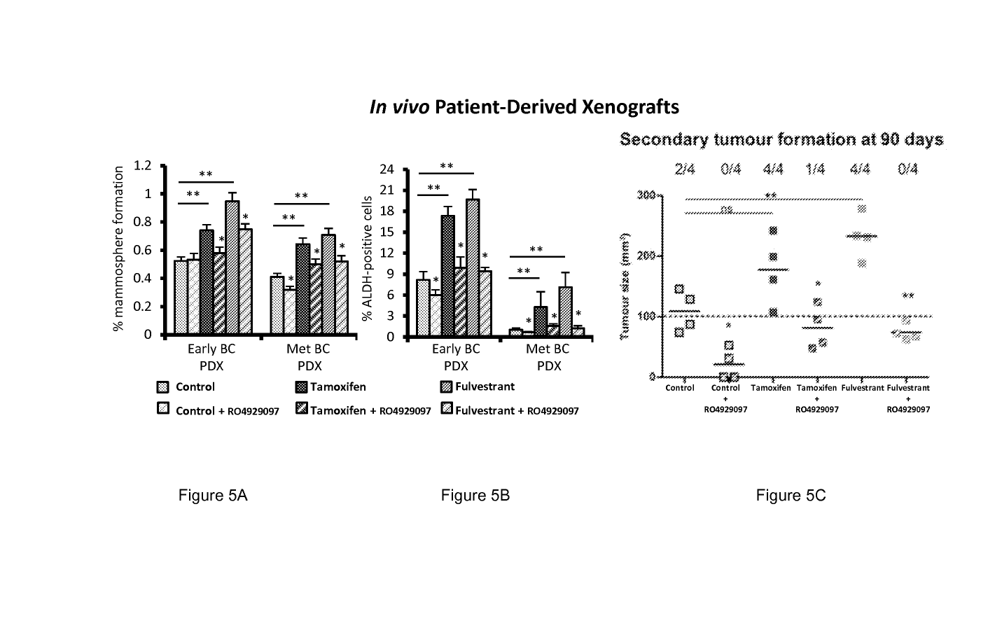

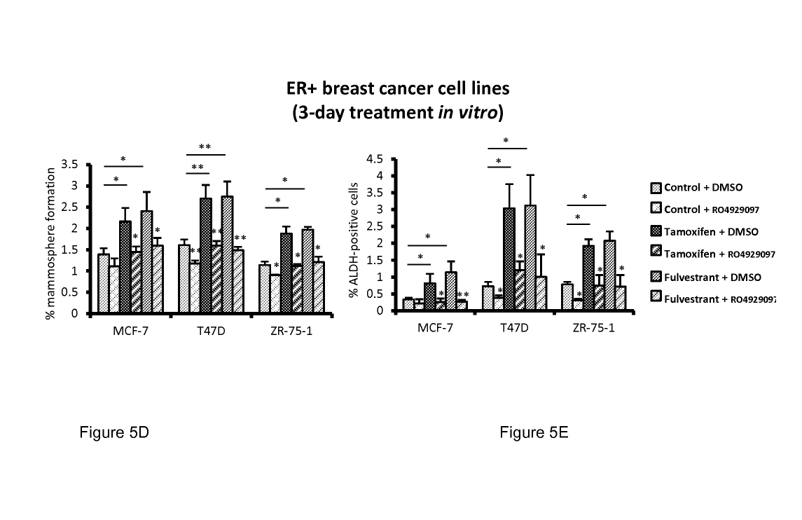

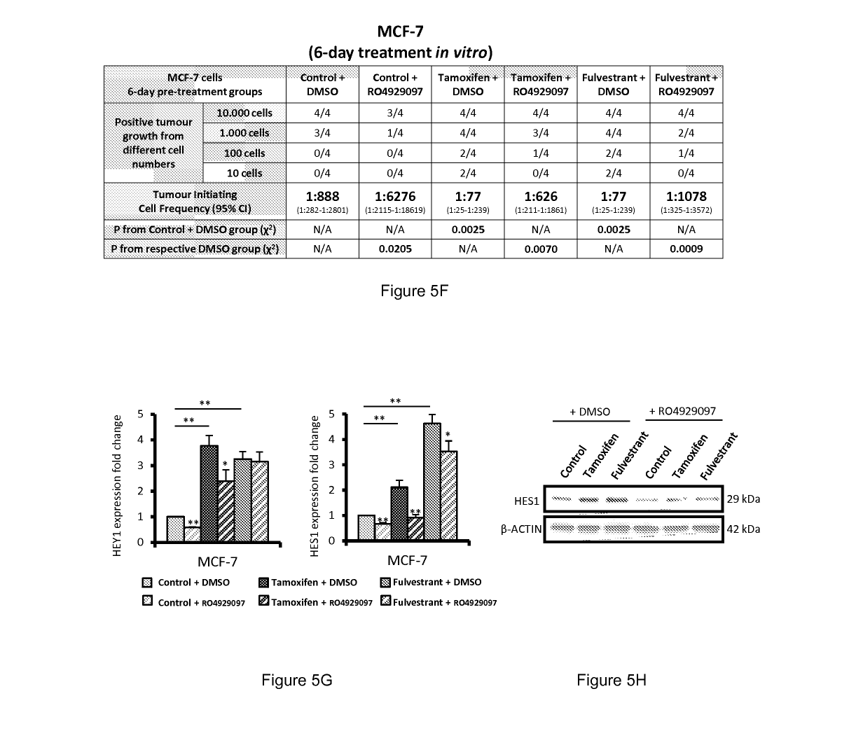

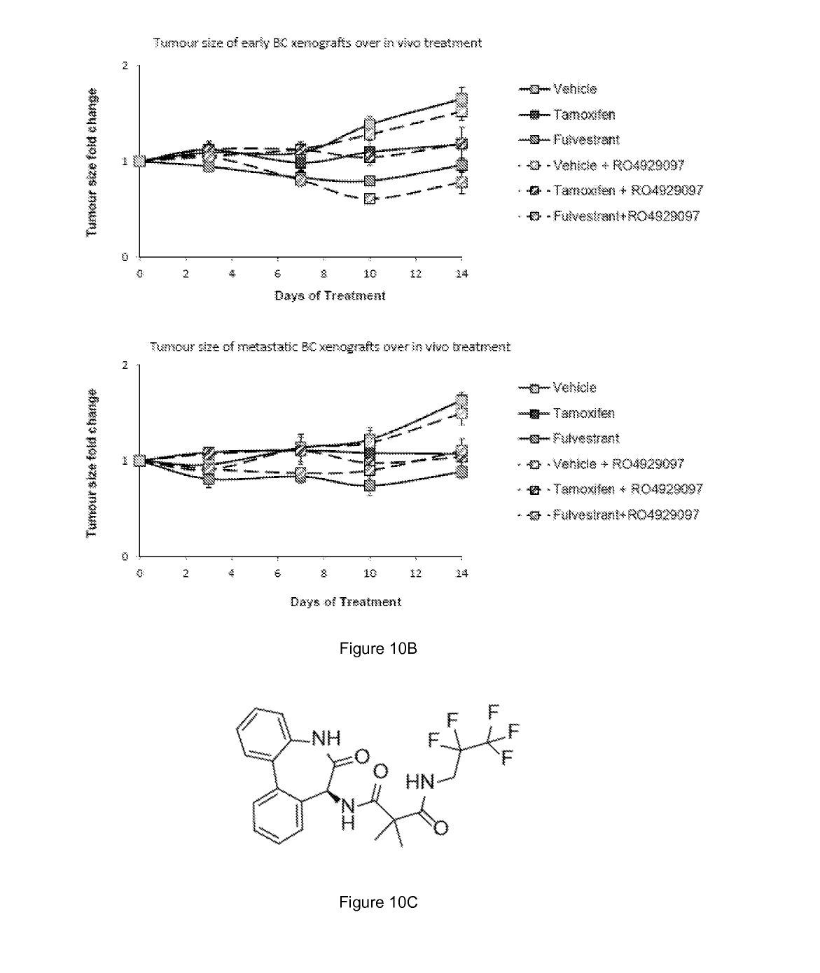

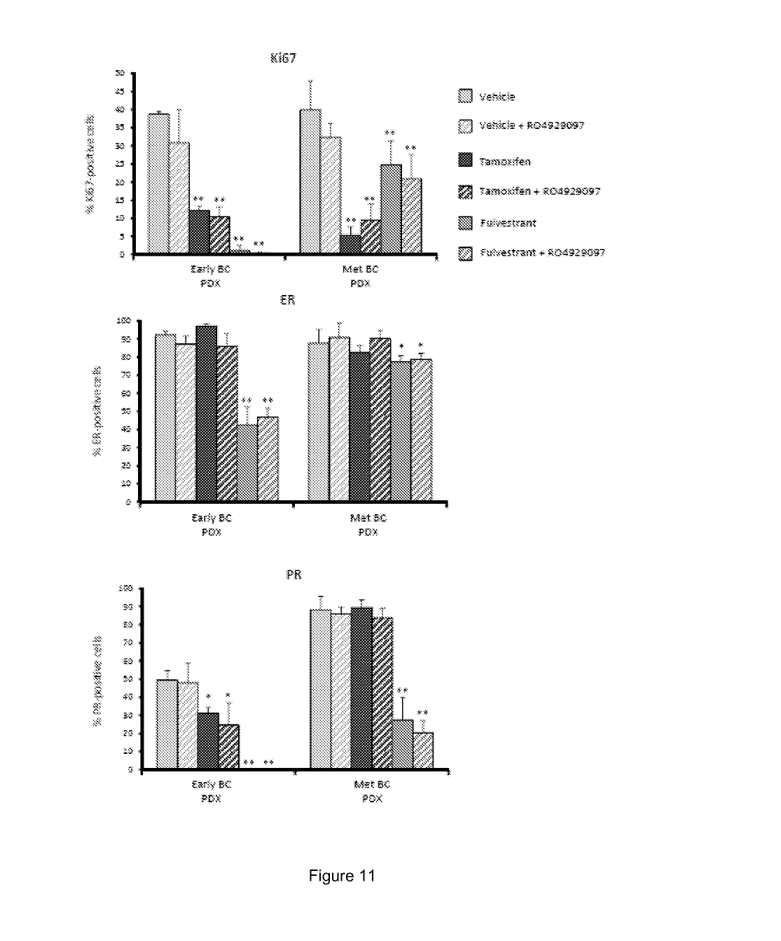

Next, we tested whether RO4929097 would abrogate BCSC activity induced in vivo by antiestrogens administered in short-term window treatments, using the same estrogen-dependent ER+ PDX tumors as in FIGS. 1D-G. Tamoxifen and fulvestrant treatments again reduced tumor growth and proliferation (FIGS. 10B and 11) whilst increasing BCSC activity assessed by both MFE and Aldefluor activity (FIGS. 5A and 5B). RO4929097 had no impact on growth or proliferation (% Ki67, FIG. 11) but significantly inhibited endocrine stimulated BCSC activity (FIGS. 5A and 5B). ER and PR protein expression levels were reduced as expected by fulvestrant treatment but were not modified by RO4929097. PR was down-regulated by tamoxifen in HBC×34 (early BC) which has not previously been exposed to anti-estrogens (FIG. 11).

The ‘gold standard’ for assaying tumor-initiating cells is xenograft formation in secondary mouse hosts which we performed using dissociated cells from PDX tumors treated in vivo with anti-estrogens and/or RO4929097. Cells isolated from tumors treated in vivo with RO4929097 had significantly reduced tumor-initiating capacity 90 days post-implantation (FIG. 5C). Furthermore, the stimulation of tumorigenicity following in vivo tamoxifen and fulvestrant treatment was completely reversed by RO4929097 (FIG. 5C). Overall, these data suggest that BCSCs surviving short-term anti-estrogen treatments have activated NOTCH4 signaling that can be blocked by combination treatment with a NOTCH4 inhibitor.

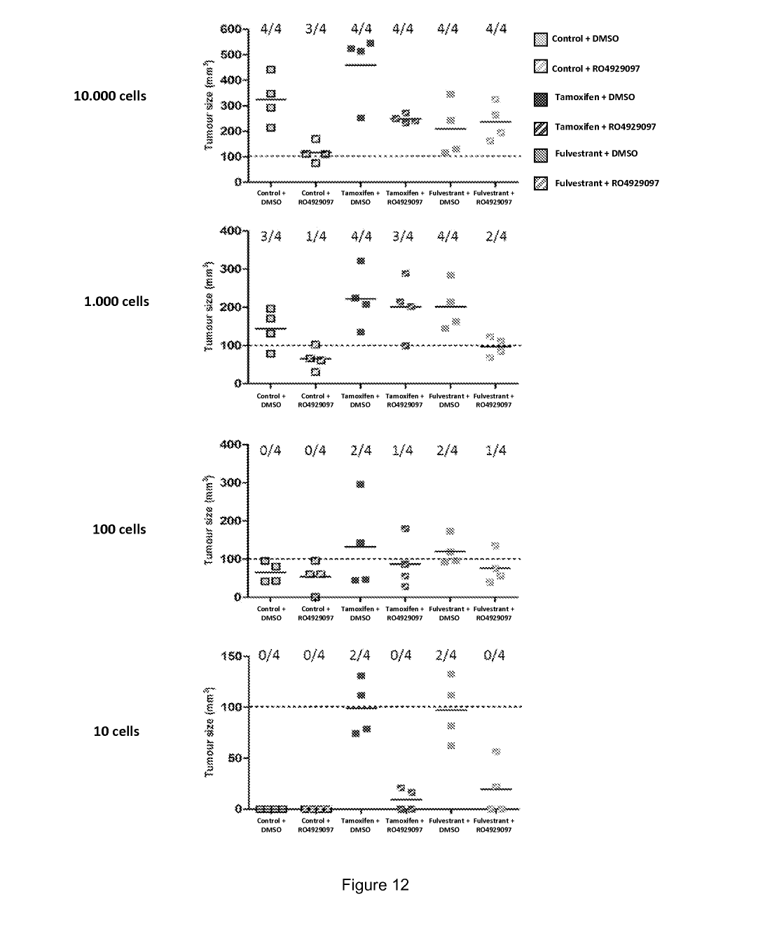

To further substantiate this finding, we analysed MFE and ALDH activity of MCF-7, T47D and ZR-75-1 cells treated for only 3 days with tamoxifen or fulvestrant concomitantly with RO4929097. In all cases RO4929097 reduced BCSC activity as assayed by MFE and ALDH-positive cells (FIGS. 5D and 5E). To confirm that RO4929097 reduced the tumor-initiating capacity, we conducted in vivo limiting dilution transplantation of MCF-7 cells. Extreme Limiting Dilution Analysis (ELDA) revealed an 11-fold enrichment in tumor-initiating cell frequency following tamoxifen or fulvestrant pre-treatment which was reversed by co-treatment with RO4929097 (FIG. 5F). FIG. 12 shows graphs representing tumor size observed in all serial dilutions. Inhibition of NOTCH4 cleavage/activation by RO4929097 was evidenced by decreased HEY1 and HES1 mRNA and protein levels (FIGS. 5G and 5H). Thus, we have established using PDX models and cell lines, both in vitro and in vivo, that NOTCH4 inhibition reduces BCSC activity induced by treatment with antiestrogens.

### NOTCH4 Inhibition Targets Cancer Stem-Like Cells in Tamoxifen-Resistant PDX Models

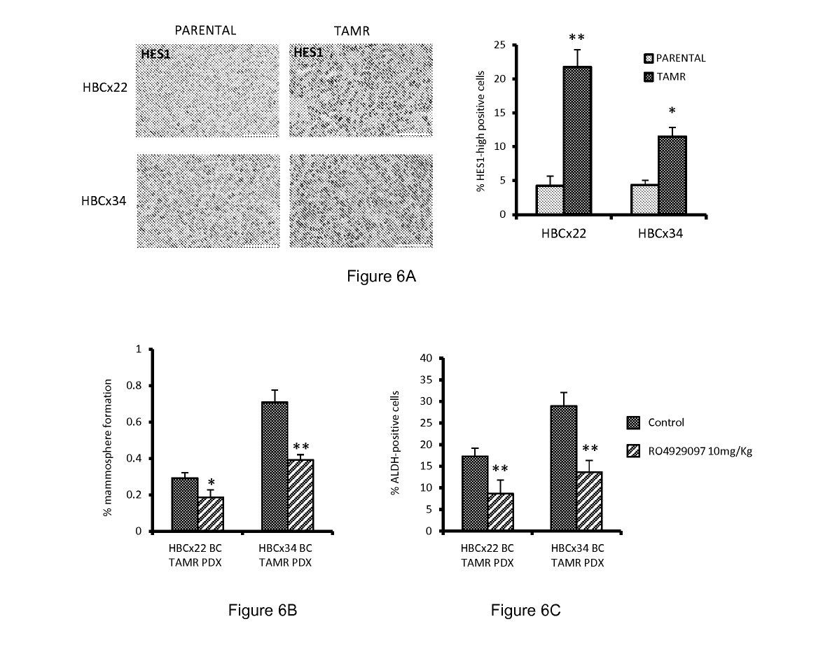

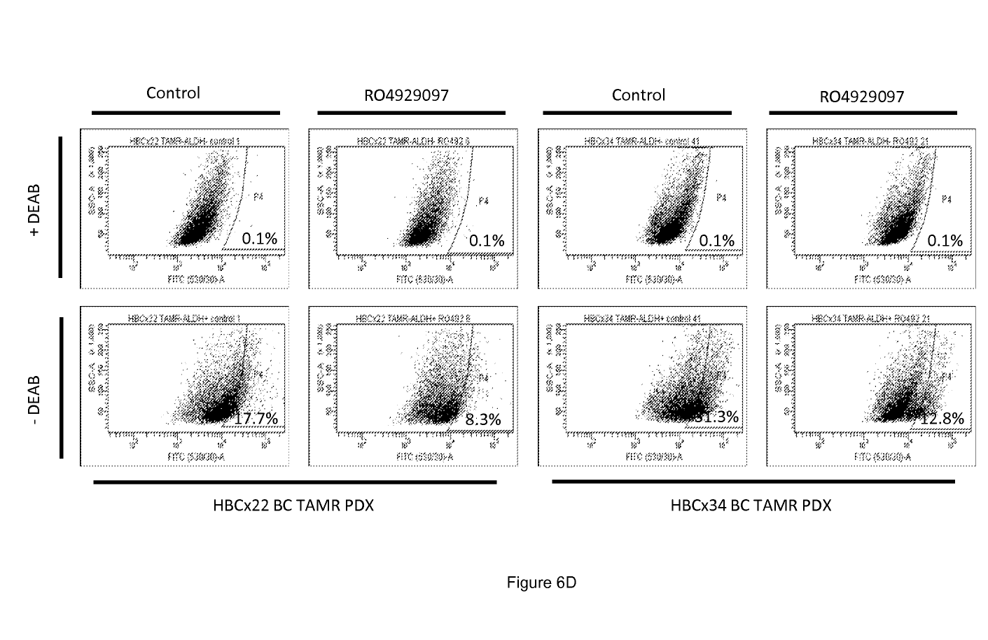

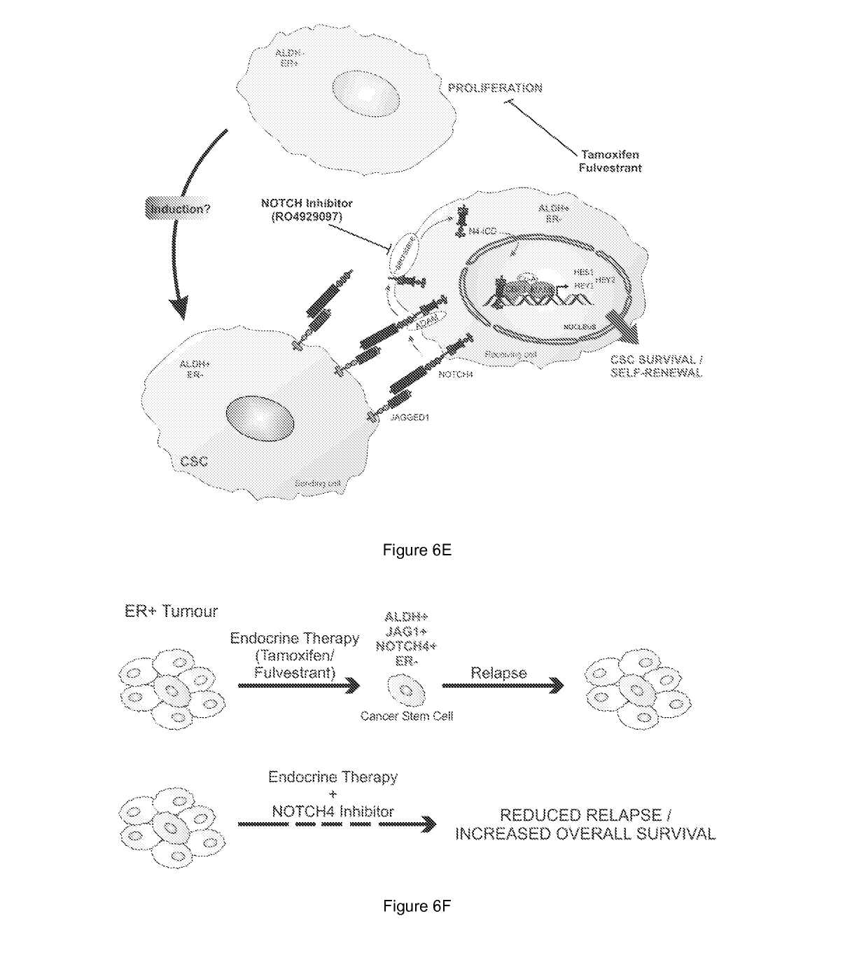

The next question we asked was whether inhibiting NOTCH4 signaling to target BCSCs will help to overcome long-term acquired antiestrogen resistance in ER+ BC patients. We therefore investigated the effects of RO4929097 treatment on BCSC activity in two established PDXs (HBC×22 and HBC×34) that have long-term acquired resistance to tamoxifen in vivo (Cottu el al, 2014). Analysis of HES1 expression by immunohistochemistry revealed that these two tamoxifen-resistant PDXs displayed increased Notch signaling activation compared to the parental control (FIG. 6A). Notably, the tamoxifen-resistant HBC×34 PDX model has a higher percentage of cells able to form mammospheres and positive for ALDH activity than the endocrine-sensitive HBC×34 PDX model (FIG. 9B). These data suggest that acquired tamoxifen resistance in PDX models also involves an enrichment of BCSCs through Notch signaling. Treatment of these tamoxifen-resistant PDX tumors in vivo with RO4929097 for 14 days demonstrates that the enrichment of BCSCs can be significantly reduced (FIGS. 6B, 6C and 6D). These data strengthen the case for therapies against NOTCH4 to target the endocrine-resistant cells responsible for relapse of ER+ tumors following hormonal therapy.

### Discussion

Resistance to antiestrogen therapy develops in 50-60% of early BC and all advanced BC (EBCTCG 2011) leading to disease relapse and death. Here we report that BCSC activity and frequency are increased in response to the common endocrine therapies tamoxifen and fulvestrant in ER+ patient samples and early and metastatic patient-derived xenografts (PDX). Previously validated in vitro and in vivo assays were used to measure BCSC activity including the mammosphere assay, Aldefluor assay and limiting dilution transplantation experiments. Our findings suggest that endocrine therapies do not target BCSCs, and this may explain how residual drug-resistant cells are responsible for the relapse of ER+ tumors following hormonal therapy. Although we observe increased BCSC frequency after endocrine treatments, we do not interpret this as an increase in absolute BCSC numbers as tamoxifen and fulvestrant are clearly successful in reducing BC recurrence in some patients. In other patients with poorer outcome after endocrine therapies, we demonstrate that tumors have high pre-treatment levels of ALDH1 expression and NOTCH4 activation. Moreover, we found that treating ER+ BC cells with endocrine therapies specifically increases NOTCH4 signaling, and that combining endocrine therapies with a Notch pathway inhibitor can prevent BCSC enrichment induced by endocrine therapies.

We demonstrate that BC cells with ALDH activity are mainly ER−, which suggests that there is an ER− population of cells in ER+ BC that would not be directly targeted by endocrine treatments. Interestingly, putative regulators of ER− BCSC activity like EGFR (Harrison et al., 2013), HER2 (Ithimakin et al., 2013) and FGFR (Fillmore et al., 2010) have previously been associated with acquisition of endocrine resistance (Hutcheson et al., 2003; Knowlden et al., 2003; McClelland et al., 2001). Here, our findings in patient-derived BCSCs add further complexity by indicating that JAG1 ligand signaling through the NOTCH4 receptor is a determining factor in the acquisition of endocrine resistance (FIG. 6E). JAG1 was recently shown to be relevant for the activation of Notch signaling in BCSCs and it has previously been associated with aggressive breast tumors and poor survival (Yamamoto et al., 2013; Reedijk et al., 2005). The activity of individual Notch receptors and interactions with different ligands may play a critical role in downstream signaling but the importance of these ligand/receptor pairings remains largely unknown.

The best described strategy for inhibition of Notch signaling is the use of small-molecule gamma-secretase inhibitors (GSI), which prevent the release of NICD. In our study, the GSI RO4929097 specifically targets NOTCH4 cleavage in antiestrogen-treated cells and thus decreases BCSC activity in vitro (MFE and ALDH activity) and tumor initiation in vivo. Consistent with these results, RO4929097 has been reported to reduce mammosphere and tumor formation of the CD44⁺CD24^{low+} BCSCs (Azzam et al., 2013). Our investigations in ER+ PDX tumors provide rationale for the use of NOTCH4 inhibitors together with endocrine therapies in the adjuvant or advanced settings (FIG. 6F). Significantly, we demonstrated the utility of RO4929097 to target BCSCs in pre-clinical models of tamoxifen-resistant patient tumors. Pilot biological investigations of endocrine agents in combination with other GSIs in window trials in BC patients have recently been reported (Albain et al., 2014), suggesting that such studies are feasible. It remains to be tested whether combining endocrine therapies and Notch pathway inhibitors can overcome resistance and prevent recurrence in vivo.

In conclusion, our data establish that tamoxifen and fulvestrant select for stem cell activity in short and long term treated BC cells as well as in early endocrine therapy naive and metastatic endocrine-treated patient-derived samples and xenografts. We provide evidence of a role for NOTCH4 signaling in the acquisition of endocrine-resistance and in the maintenance of endocrine-resistant CSCs, and our data suggest that Notch therapies may be used to prevent recurrence of ER+ tumors or to treat endocrine-resistant tumors. Finally, we report that low numbers of stem cells and low Notch signaling activation in patient tumors predict response to tamoxifen therapy and better survival. These results suggest that ER+ BC recurrence after endocrine therapies, which target the majority of cells (ER+ cells), will be reduced by targeting the NOTCH4+/ALDH1+/ER− BCSC population. In the future, translation of this knowledge into the clinical setting could result in improved outcomes for BC patients.

Anti-estrogen breast cancer therapies enrich for ALDH1 and CSCs via Notch4/Hes/Hey activation. Breast tumor CSC activity, but not growth is inhibited by GSI (RO4929097).

### Experimental Procedures

### Patient-Derived Samples

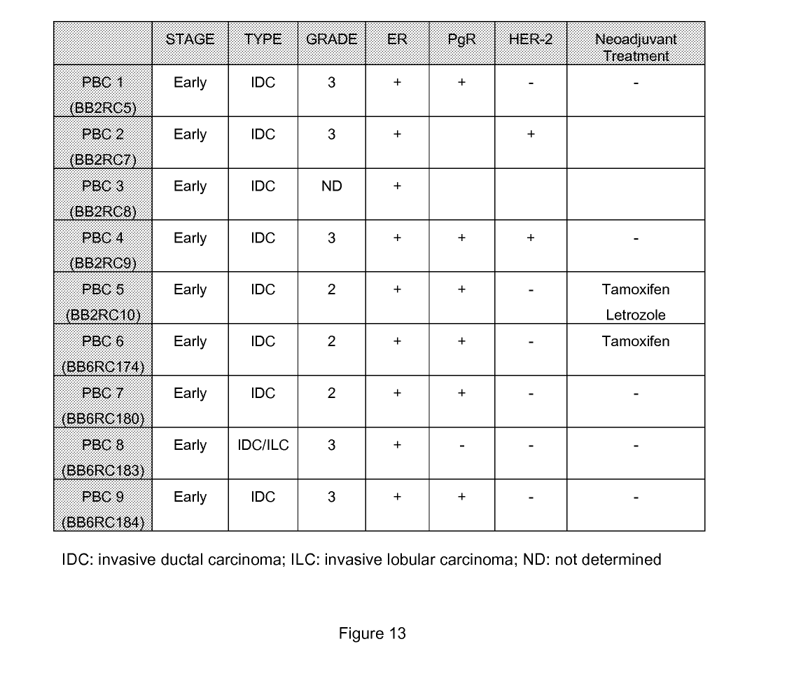

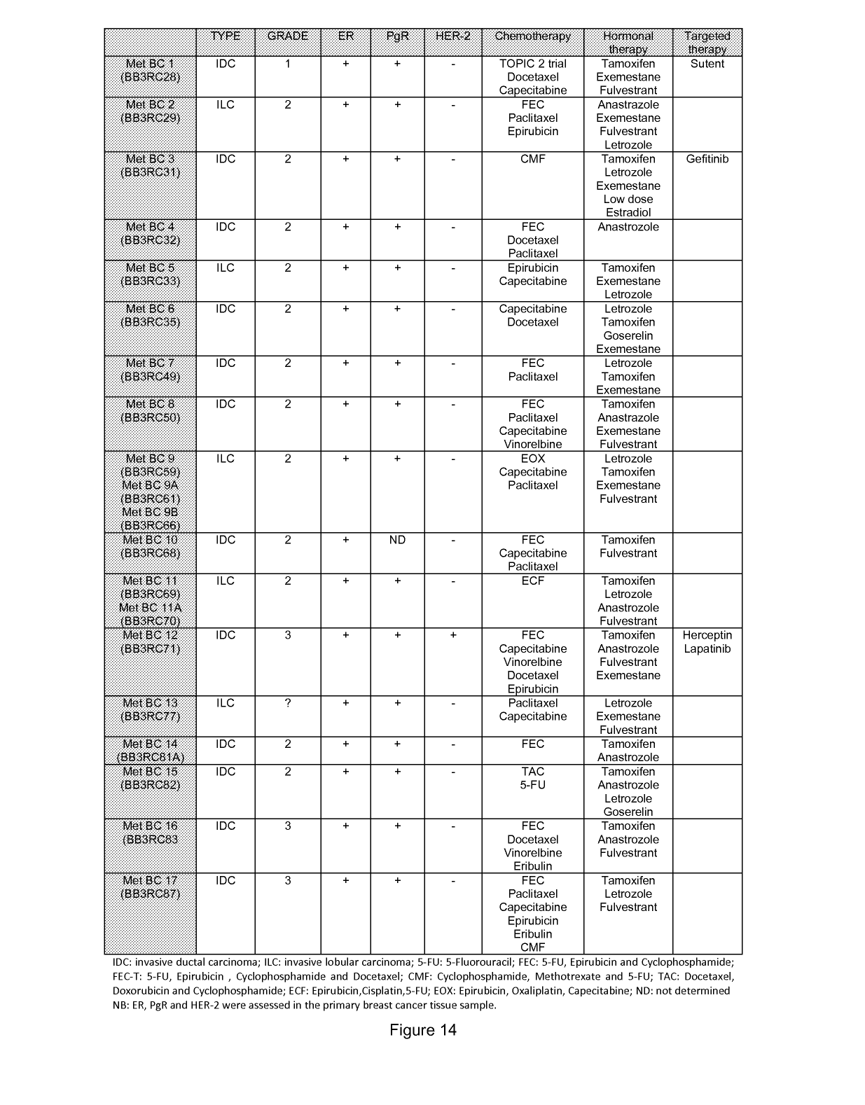

Primary human breast cancer (BC) tissue was collected from patients at South Manchester, Salford Royal and The Pennine Acute Hospitals NHS Foundation Trusts and metastatic human BC tissue was collected at The Christie NHS Foundation Trust. All patients underwent fully informed consent in accordance with local research ethics committee guidelines (study numbers: 05/Q1402/25 and 05/Q1403/159). Clinico-pathological details of the samples are summarised in FIG. 13 (primary BC) and 14 (metastatic BC).

Metastatic samples (ascites or pleural effusions) were centrifuged at 1000 g for 10 minutes at 4° C. The cell pellets were diluted in PBS. Erythrocytes and leucocytes were removed using Lymphoprep (Axis-Shield) and CD45-negative magnetic sorting (Miltenyi Biotec), respectively. Cells were cultured in adherence for 7-9 days in DMEM/F-12 medium, GlutaMAX (Gibco) with 10% FBS (Gibco), 10 μg/ml insulin (Sigma), 10 μg/ml hydrocortisone (Sigma) and 5 ng/ml epidermal growth factor (EGF, Sigma), in 10⁻⁶M 4-OH-tamoxifen (Sigma, H7904), 10⁻⁷M fulvestrant (ICI 182780, TOCRIS, 1047) or ethanol (control).

Early BC samples were collected in RPMI (Gibco), dissected into 1-2 mm³ cubes and digested with the Human Tumor Dissociation Kit (Miltenyi Biotec) for 2 hours at 37° C. Digested tissue was filtered sequentially through 100 and 40 μm cell strainers, then centrifuged at 300 g for 5 min, and washed in PBS.

### Patient-Derived Xenografts and In Vivo Experiments

Mouse studies were commenced in 8-12 week old female mice and were conducted in accordance with the UK Home Office Animal (Scientific Procedures) Act 1986. Strains used were Nude (Foxn1^{nu}) and NSG (NOD.Cg-Prkdc^{scid} Il2rg^{tm1Wjl}/SzJ) mice.

For xenografting the BB3RC31 sample a single cell suspension of 1 million tumor cells in mammosphere media (DMEM/F12 media with L-Glutamine (Gibco) containing B27 supplement (Gibco; 12587) and 20 ng/ml EGF (Sigma)) mixed 1:1 with Matrigel (BD biosciences, 356234) was inoculated subcutaneously into dorsal flanks of NSG mice in a volume of 0.2 ml. Serial passaging of the patient-derived xenograft (PDX) was carried out by implanting small fragments of the tumor subcutaneously into dorsal flanks of NSG mice. BB3RC31 is a pleural effusion from a patient who also presented with liver, lung and abdominal metastasis. For further clinico-pathological details of BB3RC31 please see Table S2.

Early (HBC×34) and metastatic (BB3RC31) BC estrogen-dependent PDXs were administered with 8 ug/ml of 17-beta estradiol in drinking water at all times and were treated with drugs when tumors reached 200-300 mm³. Experiments were performed using PDX tumors between passages 5 and 8. Animal weight and tumor size was measured bi-dimensionally using callipers twice a week. The HBC×34 model was kindly provided by Dr Elisabetta Marangoni from Institute Curie, Paris (Cottu et al., 2012).

Tamoxifen citrate (Sigma, T9262, 10 mg/kg/day) and RO4929097 (Cellagen Technology, 3 mg/kg/day) were administered by oral gavage (0.1 ml/dose) on a five day from seven basis (weekends excluded) for fourteen days. Tamoxifen citrate and RO4929097 were prepared in 1% carboxymethylcellulose (Sigma, C9481) dissolved in distilled water. Fulvestrant (kindly provided by Astrazeneca, 200 mg/kg/week) was administered by subcutaneous injection (0.1 ml/dose) on a weekly basis for fourteen days. The HBC×22 and HBC×34 tamoxifen-resistant PDXs have been established by our collaborators in the Curie Institute (Cottu el al, 2014) and were treated for fourteen days in the presence or absence of the gamma-secretase inhibitor RO4929097 (10 mg/kg/day, oral gavage). Xenografts were collected in ice cold DMEM media for live cell assays, histological analysis and RNA and protein extraction. Patient-derived xenograft single cell suspension was obtained using a collagenase-hyaluronidase mixture for digestion (Stem Cell Technologies).

To determine tumor initiation capacity of MCF-7 cells or metastatic (BB3RC31) PDX cells treated in vitro or in vivo, respectively, Nude or NSG mice were injected subcutaneously with cells in mammosphere media mixed 1:1 with Matrigel. 8 μg/ml of 17-beta estradiol was administered in drinking water or 90-day slow release estrogen pellets were implanted sub-cutaneously into mice two days before cell injection (0.72 mg, Innovative Research of America). Serial limiting dilution implantation of MCF-7 cells (10.000, 1.000, 100, 10 cells) and the Extreme Limiting Dilution Analysis (ELDA) software (The Walter and Eliza Hall Institute of Medical Research) were used to perform calculations (95% CI) of tumor initiation. Positive tumor growth was assessed at day 60 or 90 after cell injection by determining the mice bearing a tumor greater than 100 mm³ and is represented as mice positive for growth/mice tested (n=4 per condition in all experiments). p values were calculated with Chi-squared test.

### Mammosphere Colony Assay

Mammospheres are analogous to neurospheres that enrich the brain stem cells. Undifferentiated cells survive anoikis (apoptosis), self-renew and form mammospheres (Dafu et al; 2003).

Single cell suspensions of freshly isolated ER+ primary and metastatic patient-derived samples were cultured for 7-9 days (primary cells) in mammosphere colony assays in the presence of ethanol, 10⁻⁶ M 4-hydroxy-tamoxifen or 10⁻⁷ M fulvestrant. Primary mammospheres were dissociated and replated in secondary mammosphere suspension culture for a further 7-9 days to measure self-renewal. Patient-derived xenograft cells were cultured for 7-10 days. For cell lines, cells were pre-treated in adherence and then cultured for 5 days in mammosphere culture. Mammosphere formation efficiency (MFE) was calculated by dividing the number of mammospheres formed (≧50 μm) by the original number of single cells seeded (500 cells/cm² for primary cells and 200 cells/cm² for cell lines) and is expressed as fold change normalised to control or as the mean percentage of MFE (Shaw et al., 2012).

Patient-derived mammosphere cultures derived from single cells have stem cell markers, such as ALDH1+.

### Aldefluor Assay (Stemcell Technologies)

Dissociated single cells were suspended in Aldefluor assay buffer containing an ALDH substrate, bodipyaminoacetaldehyde (BAAA) at 1.5 mM, and incubated for 45 min at 37° C. To distinguish between ALDH-positive and -negative cells, a fraction of cells was incubated under identical conditions in the presence of a 2-fold molar excess of the ALDH inhibitor, diethylaminobenzaldehyde (DEAB). Mouse cells were excluded from the FACS analysis with anti-mouse MHC Class I (H-2Kd) antibody conjugated with Pacific Blue (BioLegend, 116616). 7-aminoactinomycin D (7AAD, BD) was added for dead cell exclusion. Data were acquired on a LSR II (BD) flow cytometer and analysed using the BD FACSDiva™ software.

### Tamoxifen Trial Study

Premenopausal BC patients with invasive stage II disease were enrolled in SBII:2a, a Swedish clinical trial in which patients were randomly assigned to receive 2 years of adjuvant tamoxifen or no treatment (control) and followed up for recurrence-free and overall survival (Ryden et al., 2005). Our data represents cumulative survival for a cohort of 322 pre-menopausal ER+ BC patients stratified by ALDH-low (below median) and ALDH-high (above median) expression over time.

### Notch Gene Expression Signature

The gene expression data on 669 ER+ tamoxifen-treated tumors (GSE6532, GSE9195, GSE17705, GSE12093) and 343 ER+ untreated tumors (GSE2034 and GSE7390) is from published Affymetrix microarray datasets. Gene expression data was summarised with Ensembl alternative CDF (Dai et al., 2005, Nucleic Acids Res 33:e175) and normalised with RMA, before integration using ComBat (Johnson et al., 2007, Biostatistics; 8: 118-127) to remove dataset-specific bias.

### Statistical Analysis

If not stated otherwise, a two-tailed Student's t-test was performed for statistical analysis. A value of probability (p) inferior to 0.05 was considered to be statistically significant. Error bars represent the standard error of mean (SEM) of at least three independent experiments. Data is shown as mean±SEM.

### Cell Lines and Culture

MCF7, T47D and ZR-75-1 cell lines were purchased from American Tissue Culture Collection (ATCC) and cultured in DMEM/F-12, GlutaMAX (Gibco) with 10% FBS (Gibco). Cells were treated for 3 days or 6 days with ethanol (control), 10-6M 4-OH-tamoxifen or 10⁻⁷M fulvestrant and/or with 10 μM of gamma-secretase inhibitor RO4929097 (Cellagen Technology) or DMSO (control).

MCF7 Tamoxifen- and Fulvestrant-resistant cell lines were generated by Dr Julia Gee (University of Cardiff, Wales) and were cultured in phenol red-free DMEM/F12 media with Lglutamine (Gibco) supplemented with 5% charcoal stripped serum and in the presence of 10⁻⁷M 4-OH-tamoxifen or 10⁻⁷M fulvestrant, respectively. MCF7 resistant cell lines were authenticated by comparative karyotyping to ATCC-derived MCF7.

To obtain MCF-7 overexpressing Notch intracellular domain 4 (NICD4), the pCDH-EF1-MCST2A-puro lentiviral vector (System Biosciences) was used to insert the PCR amplified human NICD4 in the multiple cloning site (MCS). GFP was cloned in place of the puromycin resistance gene. Stable expression of NICD4 and empty vector in MCF7 cells was achieved by lentiviral infection. Co-transfection of packaging vectors pPsPax2, pMD2.G together with the relevant pCDH vector in HEK293T cells was performed using Polyethylenimine (PEI) reagent (Millipore). Following the co-transfection after 8-10 hours, 10 mM sodium butyrate was used for stimulation of virus production. After 48 hours, virus containing supernatant was collected, sterile filtered and concentrated by ultracentrifugation at 25,000 rpm for 2.5 hours. 70% confluent MCF7 cells were transduced with lentiviral particles in the presence of 10 μg/ml Polybrene (Millipore). The stably infected cells were then selected based on the highest 10% expression of GFP by FACS.

MCF-7 shNotch4 cells production was previously described by Harrison and colleagues (2010). All cell lines were cultured at 37° C. in 5% CO2 and experiments were carried out at ˜70% confluence

### Cancer Tissue and Cells Analysis

Tissue microarrays (TMAs) were constructed from all formalin fixed and paraffin embedded tissue. 3×1 mm cores were taken per tissue block, and TMAs were cut into 3 μm thick sections and analysed by immunohistochemistry. Antibodies utilised were anti-ERα (Thermo, SP1), anti-PgR (Dako, M3569), anti-Ki67 (Dako, M7240), anti-HER2 (Vector Laboratories, VP-C380), anti-Hes1 (Abcam, ab108937) and anti-ALDH1 (BD Biosciences, 611195). Antigen retrieval was performed either using Target Retrieval Solution pH9 (Dako S2367, for ER, PR, Ki67 and ALDH1), Target Retrieval Solution pH6 (Dako S1699, for Hes1) or in 10 mM Citrate buffer (for HER2) in a 930 C degree water bath for 25 mins. All antibodies were detected using Dako EnVision Detection System Peroxidase/DAB, Rabbit/Mouse (Dako, K5007) and sections were counterstained with haematoxylin. Staining was quantified using Definiens Tissue Studio software. The percentage of positive epithelial cells was scored on 3 cores per tissue sample. For ER immunocytochemistry, approximately 50.000 cells were FACS sorted and then cytospun for 5 min at 80 G on poly-lysine coated slides. Cells were fixed with 4% paraformaldehyde (5 min), permeabilised with acetone (4 min) and methanol (2 min), and then blocked for 10 min with Peroxidase Blocking Reagent (Dako, S2001) and further 10 min with 3% goat serum before incubation with primary antibody. ER was then detected as described above for immunohistochemistry.

### Gene Expression Analysis Using Quantitative Real-Time PCR

Total RNA was extracted using the RNeasy Plus Mini Kit (QIAGEN, 74104) and the concentration and purity determined using an ND-1000 spectrophotometer (NanoDrop Technologies). Reverse transcription of 1 μg of RNA was performed with Oligo(dT) using the TaqMan Reverse Transcription Reagents from Applied Biosystems (N8080234). Samples were incubated on a thermal cycler (MJ Research) for 10 minutes at 25° C., 30 minutes at 48° C. and 5 minutes at 95° C. Quantitative real-time PCR reactions were set up in triplicate in 384-well plates and performed on the 7900 PCR machine (Applied Biosystems) using TaqMan® Universal PCR Master Mix (Applied Biosystems) and probes from Universal Probe Library (Roche). Conditions used for amplification of cDNA fragments were as follows: 95° C. for 5 min, 40 cycles of amplification-95 C for 15 sec, 60 C for 1 min. The expression levels were calculated using the ΔΔCt method and normalised to the housekeeping genes 36B4 and GAPDH. The sequences of the primers and probes used can be found in the table below.

| | Forward Primer | Reverse Primer | Universal Probe |
|---|---|---|---|
| GENE | 5′-3′ | 5′-3′ | Number |
| HEY1 | CGAGCTGGACGAGACCA | GAGCCGAACTCAAGTTTCCA | 39 |
| | T (SEQ ID NO: 9) | (SEQ ID NO: 10) | |
| HES1 | GAAGCACCTCCGGAACCT | GTCACCTCGTTCATGCACTC | 60 |
| | (SEQ ID NO: 11) | (SEQ ID NO: 12) | |
| 36B4 | TCTACAACCCTGAAGTGC | CAATCTGCAGACAGACACTGG | 6 |
| | TTGAT (SEQ ID NO: 13) | (SEQ ID NO: 14) | |
| GAPDH | AGCCACATCGCTCAGACA | GCCCAATACGACCAAATCC | 60 |
| | C (SEQ ID NO: 15) | (SEQ ID NO: 16) | |

### Protein Expression Analysis Using Western-Blot

Proteins were extracted with Protein Lysis Buffer (25 mM HEPES, 50 mM NaCl, 30 mM NaPP, 50 mM NaF, 1% Triton-X-100, 10% Glycerol, 5 mM EDTA, Protease Inhibitor cocktail, 1 μM PMSF). Lysates were placed on a rotator for 1 hour at 4° C., then were centrifuged at 10000 g at 4° C. for 10 min, and supernatants were collected. Protein concentrations were assessed using the BCA Protein Assay Kit (Thermo Scientific, 23225). Proteins were separated on a 10% gel (Biorad, 456-1033) by SDS-PAGE at 200V for 1 hour, and then were transferred on polyvinylidene difluoride (PVDF) membranes (BioRad, 170-4157) at 25 V/1,300 mA for 15 min using a Trans Blot Turbo (BioRad, 170-4155). Membranes were blocked in a solution of PBS containing 0.05% Tween-20 and 5% skimmed milk (Marvel) for 1 h at room temperature and primary antibodies were incubated overnight at 4° C. Primary antibodies used were anti-NOTCH1 (Rockland, 100401405), anti-NOTCH2 (Cell Signalling, D67C8), anti-NOTCH3 (Santa Cruz, sc-7424), anti-NOTCH4 (Abcam, Ab91621), anti-HES1 (Millipore, AB5702), anti-JAG1 (Santa Cruz, sc-6011), anti-JAG2 (Cell Signalling, 2210), anti-DLL1 (Abcam, Ab76655), anti-DLL3 (Abcam, Ab63707), anti-DLL4 (Abcam, Ab7280) and anti-β-actin (Sigma, A1978). Horseradish peroxidase-conjugated secondary antibodies (Dako, P0447, P0448, P0449) were incubated for 1 h at room temperature. Proteins were visualized with Luminata Classico or Luminata Forte (Millipore, WBLUC0100, WBLUF0100) by exposing the membranes to X-ray films (Hyperfilm™ MP, Amersham).

### Notch Transcriptional Assay

To measure the activation of Notch dependent transcription, cells were transfected with CBF1 firefly luciferase reporter (containing 10 copies of a CBF1 consensus sequence) and CMV-Renilla luciferase reporter. Plasmids were incubated with X-tremegene (Roche, 06366244001) in a ratio of 3:1 (μl of X-tremegene:μg of DNA) in OptiMEM (Life Technologies, 11058-021) for 15 minutes, before addition to the culture media. After 48 hours cells were lysed with 1× Passive lysis buffer (5×, Promega, E 1941), put on the rocker for 15 minutes and luminescence was assayed with the Dual-Glo Luciferase assay system (Promega, E2920) following manufacturer's instructions. Luciferase activity was measured using a luminometer (Promega, Glomax Multi+ Detection System with Instinct Software). Luminescence of the firefly luciferase was normalised to that of the renilla luciferase.

### Growth Assay (SRB Assay)

6000 cells were seeded per well in a 96 well-plate at least in triplicate for each condition used. Plates were incubated in a humidified incubator at 37° C. with 5% CO2 and an SRB assay was performed at different time points to assess cellularity. Briefly, cells were fixed with 25 μl/well of 50% (w/v) trichloroacetic (TCA) and incubated at 4° C. for a minimum of 1 hour. Fixed cells were washed 5 times with water and left to air dry. Cells were then stained with 100 μl/well of 0.4% (w/v) Sulforhodamine B (SRB) dissolved in 1% acetic acid for 30 minutes at room temperature. Residual SRB was washed away with 3 washes of 1% acetic acid and plates were left to air dry. Finally, SRB was solubilized with 100 μl/well of 10 mM Tris-base (pH 10.5) for 20 minutes at room temperature and absorbance was measured at 490 nm with an automated plate reader (BioTek ELx800).

### REFERENCES

Albain, K. S., Zlobin, A. Y., Covington, K. R., Gallagher, B. T., Hilsenbeck, S. G., Czerlanis, C. M., Lo, S., Robinson, P. A., Gaynor, E. R., Godellas, C. et al., (2014). Identification of a notch-driven breast cancer stem cell gene signature for anti-notch therapy in an ER+ presurgical window model. San Antonio Breast Cancer Symposium, Dec. 9-13, 2014, Abstract [S4-03].
Al-Hajj, M., Wicha, M. S., Benito-Hernandez, A., Morrison, S. J., and Clarke, M. F. (2003). Prospective identification of tumorigenic breast cancer cells. Proceedings of the National Academy of Sciences of the United States of America 100, 3983-3988.
Ali, S., and Coombes, R. C. (2002). Endocrine-responsive breast cancer and strategies for combating resistance. Nature reviews Cancer 2, 101-112.
Azzam, D. J., Zhao, D., Sun, J., Minn, A. J., Ranganathan, P., Drews-Elger, K., Han, X., Picon-Ruiz, M., Gilbert, C. A., Wander, S. A., et al. (2013). Triple negative breast cancer initiating cell subsets differ in functional and molecular characteristics and in gamma-secretase inhibitor drug responses. EMBO molecular medicine 5, 1502-1522.
Creighton, C. J., Li, X., Landis, M., Dixon, J. M., Neumeister, V. M., Sjolund, A., Rimm, D. L., Wong, H., Rodriguez, A., Herschkowitz, J. I., et al. (2009). Residual breast cancers after conventional therapy display mesenchymal as well as tumor-initiating features. Proceedings of the National Academy of Sciences of the United States of America 106, 13820-13825.
Cottu, P., Bieche, I., Assayag, F., El Botty, R., Chateau-Joubert, S., Thuleau, A., Bagarre, T., Albaud, B., Rapinat, A., Gentien, D., et al. (2014). Acquired resistance to endocrine treatments is associated with tumor-specific molecular changes in patient-derived luminal breast cancer xenografts. Clinical cancer research: an official journal of the American Association for Cancer Research 20, 4314-4325.
Cottu, P., Marangoni, E., Assayag, F., de Cremoux, P., Vincent-Salomon, A., Guyader, C., de Plater, L., Elbaz, C., Karboul, N., Fontaine, J. J., et al. (2012). Modeling of response to endocrine therapy in a panel of human luminal breast cancer xenografts. Breast cancer research and treatment 133, 595-606.
Dai, M., Wang, P., Boyd, A. D., Kostov, G., Athey, B., Jones, E. G., Bunney, W. E., Myers, R. M., Speed, T. P., Akil, H., et al. (2005). Evolving gene/transcript definitions significantly alter the interpretation of GeneChip data. Nucleic acids research 33, e175.
Dowsett, M., Nielsen, T. O., A'Hern, R., Bartlett, J., Coombes, R. C., Cuzick, J., Ellis, M., Henry, N. L., Hugh, J. C., Lively, T., et al. (2011). Assessment of Ki67 in breast cancer: recommendations from the International Ki67 in Breast Cancer working group. Journal of the National Cancer Institute 103, 1656-1664.
Early Breast Cancer Trialists' Collaborative, G., Davies, C., Godwin, J., Gray, R., Clarke, M., Cutter, D., Darby, S., McGale, P., Pan, H. C., Taylor, C., et al. (2011). Relevance of breast cancer hormone receptors and other factors to the efficacy of adjuvant tamoxifen: patient-level meta-analysis of randomised trials. Lancet 378, 771-784.
Farnie, G., Clarke, R. B., Spence, K., Pinnock, N., Brennan, K., Anderson, N. G., and Bundred, N. J. (2007). Novel cell culture technique for primary ductal carcinoma in situ: role of Notch and epidermal growth factor receptor signaling pathways. Journal of the National Cancer Institute 99, 616-627.
Fillmore, C. M., Gupta, P. B., Rudnick, J. A., Caballero, S., Keller, P. J., Lander, E. S., and Kuperwasser, C. (2010). Estrogen expands breast cancer stem-like cells through paracrine FGF/Tbx3 signaling. Proceedings of the National Academy of Sciences of the United States of America 107, 21737-21742.
Ginestier, C., Hur, M. H., Charafe-Jauffret, E., Monville, F., Dutcher, J., Brown, M., Jacquemier, J., Viens, P., Kleer, C. G., Liu, S., et al. (2007). ALDH1 is a marker of normal and malignant human mammary stem cells and a predictor of poor clinical outcome. Cell stem cell 1, 555-567.
Harrison, H., Farnie, G., Howell, S. J., Rock, R. E., Stylianou, S., Brennan, K. R., Bundred, N. J., and Clarke, R. B. (2010). Regulation of breast cancer stem cell activity by signaling through the Notch4 receptor. Cancer research 70, 709-718.
Harrison, H., Simoes, B. M., Rogerson, L., Howell, S. J., Landberg, G., and Clarke, R. B. (2013). Oestrogen increases the activity of oestrogen receptor negative breast cancer stem cells through paracrine EGFR and Notch signaling. Breast cancer research: BCR 15, R21.
Haughian, J. M., Pinto, M. P., Harrell, J. C., Bliesner, B. S., Joensuu, K. M., Dye, W. W., Sartorius, C. A., Tan, A. C., Heikkila, P., Perou, C. M., et al. (2012). Maintenance of hormone responsiveness in luminal breast cancers by suppression of Notch. Proceedings of the National Academy of Sciences of the United States of America 109, 2742-2747.
Hirata, N., Yamada, S., Shoda, T., Kurihara, M., Sekino, Y., and Kanda, Y. (2014). Sphingosine-1-phosphate promotes expansion of cancer stem cells via S1PR3 by a ligand-independent Notch activation. Nature communications 5, 4806.
Honeth, G., Lombardi, S., Ginestier, C., Hur, M., Marlow, R., Buchupalli, B., Shinomiya, I., Gazinska, P., Bombelli, S., Ramalingam, V., et al. (2014). Aldehyde dehydrogenase and estrogen receptor define a hierarchy of cellular differentiation in the normal human mammary epithelium. Breast cancer research: BCR 16, R52.
Hutcheson, I. R., Knowlden, J. M., Madden, T. A., Barrow, D., Gee, J. M., Wakeling, A. E., and Nicholson, R. I. (2003). Oestrogen receptor-mediated modulation of the EGFR/MAPK pathway in tamoxifen-resistant MCF-7 cells. Breast cancer research and treatment 81, 81-93.
Ithimakin, S., Day, K. C., Malik, F., Zen, Q., Dawsey, S. J., Bersano-Begey, T. F., Quraishi, A. A., Ignatoski, K. W., Daignault, S., Davis, A., et al. (2013). HER2 drives luminal breast cancer stem cells in the absence of HER2 amplification: implications for efficacy of adjuvant trastuzumab. Cancer research 73, 1635-1646.
Johnson, W. E., Li, C., and Rabinovic, A. (2007). Adjusting batch effects in microarray expression data using empirical Bayes methods. Biostatistics 8, 118-127.
Kabos, P., Haughian, J. M., Wang, X., Dye, W. W., Finlayson, C., Elias, A., Horwitz, K. B., and Sartorius, C. A. (2011). Cytokeratin 5 positive cells represent a steroid receptor negative and therapy resistant subpopulation in luminal breast cancers. Breast cancer research and treatment 128, 45-55.
Knowlden, J. M., Hutcheson, I. R., Jones, H. E., Madden, T., Gee, J. M., Harper, M. E., Barrow, D., Wakeling, A. E., and Nicholson, R. I. (2003). Elevated levels of epidermal growth factor receptor/c-erbB2 heterodimers mediate an autocrine growth regulatory pathway in tamoxifen-resistant MCF-7 cells. Endocrinology 144, 1032-1044.
Li, X., Lewis, M. T., Huang, J., Gutierrez, C., Osborne, C. K., Wu, M. F., Hilsenbeck, S. G., Pavlick, A., Zhang, X., Chamness, G. C., et al. (2008). Intrinsic resistance of tumorigenic breast cancer cells to chemotherapy. Journal of the National Cancer Institute 100, 672-679.
Lombardo, Y., Faronato, M., Filipovic, A., Vircillo, V., Magnani, L., and Coombes, R. C. (2014). Nicastrin and Notch4 drive endocrine therapy resistance and epithelial to mesenchymal transition in MCF7 breast cancer cells. Breast cancer research: BCR 16,
McClelland, R. A., Barrow, D., Madden, T. A., Dutkowski, C. M., Pamment, J., Knowlden, J. M., Gee, J. M., and Nicholson, R. I. (2001). Enhanced epidermal growth factor receptor signaling in MCF7 breast cancer cells after long-term culture in the presence of the pure antiestrogen ICI 182,780 (Faslodex). Endocrinology 142, 2776-2788.
Palmieri, C., Patten, D. K., Januszewski, A., Zucchini, G., and Howell, S. J. (2014). Breast cancer: current and future endocrine therapies. Molecular and cellular endocrinology 382, 695-723.
Patani, N., Dunbier, A. K., Anderson, H., Ghazoui, Z., Ribas, R., Anderson, E., Gao, Q., A'Hern, R., Mackay, A., Lindemann, J., et al. (2014). Differences in the transcriptional response to fulvestrant and estrogen deprivation in ER-positive breast cancer. Clinical cancer research 20, 3962-3973.
Pece, S., Tosoni, D., Confalonieri, S., Mazzarol, G., Vecchi, M., Ronzoni, S., Bernard, L., Viale, G., Pelicci, P. G., and Di Fiore, P. P. (2010). Biological and molecular heterogeneity of breast cancers correlates with their cancer stem cell content. Cell 140, 62-73.
Phillips, T. M., McBride, W. H., and Pajonk, F. (2006). The response of CD24(−/low)/CD44+ breast cancer-initiating cells to radiation. Journal of the National Cancer Institute 98, 1777-1785.
Piva, M., Domenici, G., Iriondo, O., Rabano, M., Simoes, B. M., Comaills, V., Barredo, I., Lopez-Ruiz, J. A., Zabalza, I., Kypta, R., et al. (2014). Sox2 promotes tamoxifen resistance in breast cancer cells. EMBO molecular medicine 6, 66-79.
Reedijk, M., Odorcic, S., Chang, L., Zhang, H., Miller, N., McCready, D. R., Lockwood, G., and Egan, S. E. (2005). High-level coexpression of JAG1 and NOTCH1 is observed in human breast cancer and is associated with poor overall survival. Cancer research 65, 8530-8537.
Ryden, L., Jonsson, P. E., Chebil, G., Dufmats, M., Ferno, M., Jirstrom, K., Kallstrom, A. C., Landberg, G., Stal, O., Thorstenson, S., et al. (2005). Two years of adjuvant tamoxifen in premenopausal patients with breast cancer: a randomised, controlled trial with long-term follow-up. European journal of cancer 41, 256-264.
Shaw, F. L., Harrison, H., Spence, K., Ablett, M. P., Simoes, B. M., Farnie, G., and Clarke, R. B. (2012). A detailed mammosphere assay protocol for the quantification of breast stem cell activity. Journal of mammary gland biology and neoplasia 17, 111-117.
Simões, B. M., Piva, M., Iriondo, O., Comaills, V., Lopez-Ruiz, J. A., Zabalza, I., Mieza, J. A., Acinas, O., and Vivanco, M. D. (2011). Effects of estrogen on the proportion of stem cells in the breast. Breast cancer research and treatment 129, 23-35.
Stylianou, S., Clarke, R. B., and Brennan, K. (2006). Aberrant activation of notch signaling in human breast cancer. Cancer research 66, 1517-1525.
Yamamoto, M., Taguchi, Y., Ito-Kureha, T., Semba, K., Yamaguchi, N., and Inoue, J. (2013). NF-kappaB non-cell-autonomously regulates cancer stem cell populations in the basal-like breast cancer subtype. Nature communications 4, 2299.
Yun, J., Pannuti, A., Espinoza, I., Zhu, H., Hicks, C., Zhu, X., Caskey, M., Rizzo, P., D'Souza, G., Backus, K., et al. (2013). Crosstalk between PKCalpha and Notch-4 in endocrine-resistant breast cancer cells. Oncogenesis 2, e60.
Zardavas, D., Baselga, J., and Piccart, M. (2013). Emerging targeted agents in metastatic breast cancer. Nature reviews Clinical oncology 10, 191-210.
Zhao, D., Mo, Y., Li, M. T., Zou, S. W., Cheng, Z. L., Sun, Y. P., Xiong, Y., Guan, K. L., and Lei, Q. Y. (2014). NOTCH-induced aldehyde dehydrogenase 1A1 deacetylation promotes breast cancer stem cells. The Journal of clinical investigation 124, 5453-5465.

1. A method comprising determining the level of ALDH1 in a subject with estrogen receptor positive breast cancer and selecting the subject of treatment with an inhibitor of Notch Signaling (INS) if the level of ALDH1 is elevated.

2. The method according to claim 1 further comprising administering endocrine therapy to the subject.

3. The method according to claim 1 wherein the subject has exhibited resistance to endocrine therapy.

4. The method according to claim 1 wherein the subject has late stage breast cancer.

5. The method according to claim 1 wherein the INS is a gamma secretase inhibitors (GSI).

6. The method according to claim 1 wherein the subject is undergoing endocrine therapy.

7. The method according to claim 6 wherein the subject is undergoing therapy with tamoxifen or fulvestrant or tamoxifen and fulvestrant.

8. The method according to claim 4 wherein the subject has metastatic breast cancer.

9. The method according to claim 1 wherein the subject is selected for treatment of ALDH1 level is elevated relative to a subject known to be responsive to endocrine therapy.

10. The method according to claim 1 wherein the subject is selected for treatment if ALDH1 level is elevated relative to the level of ALDH1 in the same subject at an earlier date.

11. The method according to claim 6 wherein the level of ALDH1 is elevated compared to the level of ALDH1 in the patient prior to undergoing endocrine drug therapy.

12. The method according to claim 1 wherein the GSI is RO4929097.

13. A method comprising selecting a subject undergoing endocrine therapy to treat ER+ breast cancer; determining the level of ALDH1 in the subject; and selecting the subject for treatment with INS if the level of ALDH1 is determined to be elevated.

14. The method according to claim 13, further comprising administering INS to the patient.

15. A method for predicting the outcome of hormone breast cancer therapy in a subject, the method comprising determining the level of ALDH1 in the subject, high level of ALDH1 indicates a poor response to treatments with a SERM or SERD drug, and selecting the subject for treatment with breast cancer therapy which is not a SERM or SERD drug, or selecting the subject for treatment with a INS in addition to the SERM or SERD drugs.

16. A method of treatment comprising administering to a subject determined to have ER+ breast cancer and elevated ALDH1, an effective amount of an endocrine therapy and a INS.

17. A method of treatment comprising administering endocrine therapy and an INS to a subject determined to have ER+ breast cancer that is resistant to endocrine therapy.

18. A method comprising determining the level of expression of one or more biomarker selected from NOTCH4, HES1, HEY1 and HEY2 in a subject with ER+ breast cancer and, if said one or more genes is elevated, selecting the patient for treatment for combination therapy with an INS and an endocrine therapy.

19. A pharmaceutical composition comprising a SERD or SERM and a INS.

20. The pharmaceutical composition according to claim 19 wherein the SERM is tamoxifen.

21. The pharmaceutical composition according to claim 19 wherein the SERM is fulvestrant.

22. The pharmaceutical composition according to claim 19 wherein the INS is RO49290967 or a derivative thereof.
